(19) United States
(12) Patent Application Publication
(10) Pub. No.: US 2022/0298490 A1
(43) Pub. Date: Sep. 22, 2022

(54) METHOD TO GENERATE MONOCYTIC PROGENITOR CELLS

(71) Applicant: Hoffmann-La Roche Inc., Little Falls, NJ (US)

(72) Inventors: Nadine DAHM, Bad Bellingen (DE); Simon GUTBIER, Konstanz (DE); Christoph PATSCH, New York, NY (US)

(73) Assignee: Hoffmann-La Roche Inc., Little Falls, NJ (US)

(21) Appl. No.: 17/520,267

(22) Filed: Nov. 5, 2021

Related U.S. Application Data
(63) Continuation of application No. PCT/EP2020/064481, filed on May 26, 2020.

(30) Foreign Application Priority Data
May 28, 2019 (EP) 19176852.2

(51) Int. Cl. C12N 5/074; C12N 5/0735

(52) U.S. Cl. CPC C12N 5/0696; C12N 5/0606; C12N 2501/155; C12N 2501/115; C12N 2506/02; C12N 2500/90; C12N 2502/08; C12N 2506/45

(57) ABSTRACT

This application relates to methods for generating monocytic progenitor cells and their differentiation into macrophages and microglia as well as to large scale cell cultures for producing monocytic progenitor cells.

### FIELD OF THE INVENTION

This application relates to methods for generating monocytic progenitor cells and their differentiation into macrophages and microglia as well as to large scale cell cultures for producing monocytic progenitor cells.

### BACKGROUND

Monocytes and macrophages are key players in inflammatory processes and their activation and functionality is crucial in health and disease (Biswas et al. 2012; Mantovani et al. 2013; Sica et al. 2008; Wynn et al. 2013). The diseases with confirmed macrophage involvement encompass metabolic diseases, allergic disorders, autoimmunity, cancer, neurodegenerative diseases as well as bacterial, viral, parasitic and fungal infections. Beside the mediation of the acute immune-defense in a disease context, macrophages, which are widely distributed throughout tissues, are essential in repair and homeostasis of the surrounding tissue. Therefore, impaired macrophage functionality and the subsequent loss of homeostasis are closely linked to the pathogenesis of degenerative diseases.

Key macrophage functions in homeostasis and disease defense include phagocytosis (pathogens, debris and dead cells), migration (to the side of damage) as well as cytokine release to trigger further inflammatory responses or render trophic support to the surrounding tissue. (Biswas et al. 2012; Mantovani et al. 2013; Sica et al. 2008; Wynn et al. 2013). For this reason, the modulation of monocyte/macrophage function reflects a therapeutic strategy to possibly resolve many diseases. The broad range of disease areas with macrophage involvement and functional properties of macrophages results in a huge variety of potential targets (Tiwari et al. 2008). This generates a high demand of monocytes and macrophages for drug development and screening.

Until now, macrophage research has been complicated and slowed down by limitations in the generation of relevant cells. One way, which was mainly used in the past, to obtain macrophages, is the isolation of monocytes from PBMCs (peripheral blood mononuclear cells) concentrated from blood donation (FIG. 1). However, limited cell numbers per donor, donor-to-donor variation and confined, genetic engineering possibilities restrict the use of these primary cells.

Recent studies successfully derived monocytic progenitor cells and macrophages from iPS cells (Ackermann et al. 2018; Hong et al. 2018; Karlsson et al. 2008; Senju et al. 2011; Takamatsu et al. 2014; van Wilgenburg et al. 2013). This approach has several advantages compared to the isolation of primary monocytes (FIG. 1). It allows the use of cells with a disease relevant genetic background, genetic engineering (i.e. correction of a disease causing mutation in the pluripotent state) and limits the donor variability where needed. iPS technology offers a virtually unlimited supply of monocytes/macrophages of consistent genotype and function.

Microglia are special subtype of tissue resident macrophages. During embryonic development two waves of macrophages are produced in blood islands of the yolk sac. These yolk sac derived macrophages are Myb independent but dependent on PU.1 and IRF8 (Haenseler et al. 2016) for proliferation and give rise to tissue resident macrophages. While in a lot of tissues this initial macrophage population gets partially or entirely replaced by bone-marrow derived macrophages, the brain resident macrophage population, the microglia, is still solely of that origin.

Microglia have important homeostatic functions, such as clearance of misfolded proteins and dead cells, pruning of synapses and releasing neurotrophic factors. Moreover, upon inflammatory stimulation they can become activated and release potentially harmful cytokines and produce reactive oxygen species. Chronic inflammatory activation and the expression of high levels of several genetic risk factors for neurodegenerative diseases (such as LRRK2, TREM2, ASYN and CD33) created high interest on the role of microglia in neurodegenerative diseases and neuroinflammation.

Until now, due to the poor availability of human primary microglia and relevant human cell models, microglia research has been limited to primary rodent cells. Recent protocols (Abud et al. 2017; Ackermann et al. 2018; Brownjohn et al. 2018; Douvaras et al. 2017; Haenseler et al. 2017a; Haenseler et al. 2017b; Hong et al. 2018; Karlsson et al. 2008; Muffat et al. 2016; Senju et al. 2011; Takamatsu et al. 2014; van Wilgenburg et al. 2013) generating monocytes and macrophages from iPS cells showed the correct ontogeny markers and the generation of microglia like cells from that precursors in a neuronal co-culture has been described recently (Haenseler et al. 2017a).

However, the protocols provided by the referenced literature are limited in throughput and stability of cell cultures and, therefore, cannot provide the amount of cells qualitatively and quantitative needed for high-throughput assays, e.g., in drug discovery and development.

Hence, there remains a need for improved protocols for generating large amounts of monocytic progenitor cells from iPS cells in high-throughput mode.

### SUMMARY OF THE INVENTION

Provided is a method for producing monocytic progenitor cells, the method comprising the step of:

a) plating pluripotent stem cells in a pluripotency medium on a cell culture support coated with laminin;

b) harvesting the pluripotent stem cells and contacting the pluripotent stem cells with a mesoderm induction medium in suspension culture;

c) plating the cells on a cell culture support suitable for attachment of the cells; and

d) harvesting monocytic progenitor cells from the cell culture supernatant.

In one embodiment, the laminin in step a) comprises the laminin subunit alpha-5, in particular wherein the laminin in step a) comprises the laminin subunits alpha-5, beta-2 and gamma-1.

In one embodiment, the cells are contacted in step b) with a defined medium comprising BMP4.

In one embodiment, the cells are contacted in step b) with a defined medium comprising VEGF.

In one embodiment, the cells are contacted in step b) with a defined medium comprising SCF.

In one embodiment, the cells in step b) form embryoid bodies (EBs).

In one embodiment, the cell culture support in step c) is coated with a basement membrane biomaterial.

In one embodiment, the cells in step c) are contacted with a myeloid maturation medium.

In one embodiment, the myeloid maturation medium comprises M-CSF.

In one embodiment, the myeloid maturation medium comprises IL-3.

In one embodiment, the method further comprises step e) differentiating the harvested monocytic progenitor cells into macrophages.

In one embodiment, the cells in step e) are plated onto a non-coated tissue culture support.

In one embodiment, the method further comprises step e) differentiating the harvested monocytic progenitor cells into microglia.

Further provided is an adherent large scale cell culture for producing monocytic progenitor cells, wherein the adherent cell culture is capable of producing at least about 100'000 monocytic progenitor cells per cm² of cell culture area per week.

### SHORT DESCRIPTION OF THE FIGURES

FIG. 1: Schematic depiction of methods to derive monocytic progenitors and macrophages from induced pluripotent stem cells (iPSCs). Adult donor cells can be re-programmed to generate iPSCs. Using the right combination of differentiation cues (cytokines, morphogens, growth factors and small molecules) cellular lineage development can be directed in vitro and used to generate the desired cell type (i.e. macrophages). This way offers unlimited supply of cells from a single donor and allows the use of cells from donors with a disease specific genetic background. Furthermore, iPSCs can genetically be modified and clonally selected in the self-renewing pluripotent state. This technique allows the generation of isogenic iPSC lines and its cellular derivate (e.g. macrophages) can be directly compared to the respective health or diseased parental iPSC clone. An alternative way to obtain monocytes and macrophages is the isolation from human blood donations. Cells obtained in this way are limited in their number per donor and due to their postmitotic state the generation of genetically modified clonal lines is not feasible. Further variation can arise from various donor conditions (physiological state), such as infections prior to blood donation.

FIG. 2: Schematic depiction of sequential differentiation steps in the generation process of iPSC derived macrophages. iPSCs are cultured and maintained in the pluripotent state (Step 1). When passaging the maintenance culture 2-10 million iPSCs are used to initiate embryoid bodies (EBs) formation (Step 2). After 4 days of EB formation, the pre-differentiated EBs are plated on cell-culture dishes and form blood factories in the subsequent time period (Step 3). Blood factories start to produce and release the first monocytic progenitors as soon as 14 days after start of differentiation. These progenitors can be harvested twice per week from the supernatant up to more than 100 days. Monocytic progenitors are further differentiated for 7 days to macrophages (Step 4), depending on experimental prerequisites these macrophages can be polarized further by cytokine addition to give rise to specific inflammatory or regulatory subtypes (Step 5).

FIG. 3: Schematic depiction of differentiation timeline. Cytokines, growth factors, morphogens, media and coating used in the 5 sequential differentiation steps (Steps 1-5) are as indicated.

FIGS. 4A-D: Comparison of new culture conditions with the previously published method of Wilgenburg et al. (2013). iPSCs were either cultured on growth-factor reduced matrigel or Laminin-521, blood factories were differentiated as depicted in FIGS. 2 and 3 and compared at day 21 of differentiation.

FIG. 4A: Blood factories (adherent cells) derived from iPSCs cultured on Laminin-521 produce monocytic progenitors already at day 21 of differentiation.

FIG. 4B: Monocytic progenitors (non-adherent cells) in the supernatant of the blood factories derived from iPSCs cultured on Laminin-521 at day 21 of differentiation.

FIG. 4C: Blood factories (adherent cells) derived from iPSCs cultured on matrigel do not produce monocytes at day 21 of differentiation.

FIG. 4D: Few monocytic progenitors (non-adherent cells) in the supernatant of the blood factories derived from iPSCs cultured on Matrigel at day 21 of differentiation.

FIGS. 5A-B: Comparison of new culture conditions with the previously published method of Wilgenburg et al. (2013). iPSCs were either cultured on matrigel or Laminin-521, blood factories were differentiated as depicted in FIGS. 2 and 3 and compared at day 21 of differentiation. Monocytic progenitors derived from iPSCs cultured on Laminin-521 were analyzed by flow cytometer for myeloid markers CD14 and CD11b.

FIG. 5A: Flow cytometry dot plot analysis of CD11b surface staining of monocytic progenitors harvested from Laminin-521 derived cultures and isotype control. Multiple peaks indicate inhomogeneous CD11b positive cell population.

FIG. 5B: Flow cytometry dot plot analysis of CD14 surface staining of monocytic progenitors harvested from Laminin-521 derived cultures and isotype control. Multiple peaks indicate inhomogeneous CD14 positive cell population.

FIGS. 6A-H: Comparison of new culture conditions with the previously published method of Wilgenburg et al. (2013). iPSCs were either cultured on Matrigel or Laminin-521, blood factories were differentiated as depicted in FIGS. 2 and 3 and monocytic progenitors were collected from the supernatant and compared at day 34 of differentiation. Monocytic progenitors derived from iPSCs cultured either on Laminin-521 or matrigel were analyzed for myeloid markers CD14, CD11b, CD68 and proliferation marker Ki67 by flow cytometer. Average yield from a B10 culture dish was 36.5*10⁶ viable cells for Laminin-521 derived cultures and 1.2*10⁶ viable cells for matrigel derived cultures.

FIG. 6A: Flow cytometry dot plot analysis of CD11b surface staining of monocytic progenitors harvested from Laminin-521 derived cultures at day 34 and isotype control. Single peak indicates homogeneous CD11b positive cell population.

FIG. 6B: Flow cytometer dot plot analysis of CD14 surface staining of monocytic progenitors harvested from Laminin-521 derived cultures at day 34 and isotype control. Single peak indicates homogeneous CD14 positive cell population.

FIG. 6C: Flow cytometer dot plot analysis of CD68 staining of monocytic progenitors harvested from Laminin-521 derived cultures at day 34 and isotype control. Single peak indicates homogeneous CD68 positive cell population.

FIG. 6D: Flow cytometry dot plot analysis of Ki67 proliferation marker of monocytic progenitors harvested from Laminin-521 derived cultures at day 34 and isotype control. Single peak at the intensity of the isotype control indicates low proliferative activity in the cell population.

FIG. 6E: Flow cytometry dot plot analysis of CD11b surface staining of monocytic progenitors harvested from matrigel derived cultures at day 34 and isotype control. Single peak indicates homogeneous CD11b positive cell population.

FIG. 6F: Flow cytometry dot plot analysis of CD14 surface staining of monocytic progenitors harvested from Matrigel derived cultures at day 34 and isotype control. Single peak indicates homogeneous CD14 positive cell population.

FIG. 6G: Flow cytometry dot plot analysis of CD68 staining of monocytic progenitors harvested from matrigel derived cultures at day 34 and isotype control. Single peak indicates homogeneous CD68 positive cell population.

FIG. 6H: Flow cytometry dot plot analysis of Ki67 proliferation marker of monocytic progenitors harvested from matrigel derived cultures at day 34 and isotype control. Single peak at the intensity of the isotype control indicates low proliferative activity in the cell population.

FIGS. 7A-H: Comparison of new culture conditions with the previously published method of Wilgenburg et al. (2013). iPSCs were either cultured on matrigel or Laminin-521, blood factories were differentiated as depicted in FIGS. 2 and 3 and monocytic progenitors were collected from the supernatant and compared at day 41 of differentiation. Monocytic progenitors derived from iPSCs cultured either on Laminin-521 or matrigel were analyzed for myeloid markers CD14, CD11b, CD68 and proliferation marker Ki67 by FACS analysis. Average yield from a B10 culture dish was 30*10⁶ viable cells for Laminin-521 derived cultures and 8.5*10⁶ viable cells for matrigel derived cultures.

FIG. 7A: Flow cytometry dot plot analysis of CD11b surface staining of monocytic progenitors harvested from Laminin-521 derived cultures at day 41 and isotype control. Single peak indicates homogeneous CD11b positive cell population.

FIG. 7B: Flow cytometry dot plot analysis of CD14 surface staining of monocytic progenitors harvested from Laminin-521 derived cultures at day 41 and isotype control. Single peak indicates homogeneous CD14 positive cell population.

FIG. 7C: Flow cytometry dot plot analysis of CD68 staining of monocytic progenitors harvested from Laminin-521 derived cultures at day 41 and isotype control. Single peak indicates homogeneous CD68 positive cell population.

FIG. 7D: Flow cytometry dot plot analysis of Ki67 proliferation marker of monocytic progenitors harvested from Laminin-521 derived cultures at day 41 and isotype control. Single peak at the intensity of the isotype control indicates low proliferative activity in the cell population.

FIG. 7E: Flow cytometry dot plot analysis of CD11b surface staining of monocytic progenitors harvested from matrigel derived cultures at day 41 and isotype control. Single peak indicates homogeneous CD11b positive cell population.

FIG. 7F: Flow cytometry dot plot analysis of CD14 surface staining of monocytic progenitors harvested from matrigel derived cultures at day 41 and isotype control. Single peak indicates homogeneous CD14 positive cell population.

FIG. 7G: Flow cytometry dot plot analysis of CD68 staining of monocytic progenitors harvested from matrigel derived cultures at day 41 and isotype control. Single peak indicates homogeneous CD68 positive cell population.

FIG. 7H: Flow cytometry dot plot analysis of Ki67 proliferation marker of monocytic progenitors harvested from matrigel derived cultures at day 41 and isotype control. Single peak at the intensity of the isotype control indicates low proliferative activity in the cell population.

FIG. 8: Comparison of new culture conditions with the previously published method of van Wilgenburg et al. (2013). IPSCs were either cultured on matrigel (van Wilgenburg et al. 2013) or Laminin-521, blood factories were differentiated as depicted in FIGS. 2 and 3 and monocytic progenitors were collected from the supernatant and compared at day 21; 35; 41 of differentiation. Monocyte yield and marker expression for the different harvest days is summarized. Blood factories derived from IPSC grown on Laminin-521 mature, produce, release faster and with greater yield monocytic progenitors in the supernatant.

FIGS. 9A-B: Comparison of new culture conditions with the previously published method of Wilgenburg et al. (2013). IPSCs were either cultured on matrigel (van Wilgenburg et al. 2013) or Laminin-521, blood factories were differentiated as depicted in FIGS. 2 and 3 and monocytic progenitors were collected from the supernatant and compared at every 7 days starting at day 27 of differentiation up to day 111 of differentiation. Monocytic progenitors derived from iPSCs cultured either on Laminin-521 (9A) or matrigel (9B) were analyzed for myeloid markers CD14, CD11b, CD68 and proliferation marker Ki67 by FACS analysis.

FIG. 10: Comparison of iPSC derived monocytes with CD14+ monocytes isolated from PBMCs. Monocytes from both sources were analyzed for myeloid markers CD14, CD11b, CD68 and proliferation marker Ki67 by FACS analysis. Cell types from both sources express CD14, CD11b, CD68 and are Ki67 negative. Intensity of the markers differs between cells from the two sources pointing to slight differences in the amount of CD14, CD11b and CD68 respectively.

FIG. 11: Comparison of iPSC derived macrophages with CD14+ monocytes isolated from PBMC derived macrophages. Monocytes from both sources were differentiated as described in materials and methods and analyzed for myeloid markers CD14, CD11b, CD68 and proliferation marker Ki67 by FACS analysis at day 7 of macrophage differentiation. Cell types from both sources express CD14, CD11b, CD68 and are Ki67 negative. Intensity of the markers differs between cells from the two sources pointing to slight differences in the amount of CD14, CD11b and CD68 respectively.

FIGS. 12A-F: Comparison of new culture conditions with the previously published method of van Wilgenburg et al. (2013). Embryoid bodies generated from three different iPSC lines, SFC840 (FIGS. 12A and 12D), Gibco episomal (FIGS. 12B and 12E) and SA001 (FIGS. 12C and 12F), were either plated on uncoated cell culture dishes (12A-C) or on growth factor reduced (GFR) matrigel coated culture dishes (12D-F). Adherence of and cell outgrowth from embryoid bodies was better on GFR matrigel for all the three tested cell-lines, ensuring a more robust culture development. The cell layer prevent monocytic progenitors from adhering to the surface of the tissue culture dishes further increasing the number of monocytic progenitors in the supernatant.

FIGS. 13A-F: Comparison of new culture conditions with the previously published method of van Wilgenburg et al. (2013). Embryoid bodies generated from three different iPSC lines, SFC840 (FIGS. 13A and 13D), Gibco episomal (FIGS. 13B and 13E) and SA001 (FIGS. 13C and 13F), were either plated on uncoated cell culture dishes (13A-C) or on growth factor reduced (GFR) matrigel coated culture dishes (13D-F). More monocytic progenitors are released in the supernatant by blood factories generated by embryoid bodies grown on GFR matrigel compared to uncoated dishes already at day 21 of differentiation.

FIG. 14: Monocytic progenitors originating from 3 different cell lines (SFC840-03-01, SA001 and Gibco episomal) were differentiated for 7 days into macrophages as described in material and methods. Phagocytosis assay was performed, by feeding Alexa488 labeled zymosan particles to the 3 different iPSC-derived macrophage lines. After 1 hour of phagocytosis, cells were detached and Alexa488 positive cells were measured by flow cytometer. Macrophages originating from all sources displayed strong phagocytic capability ranging from 50% to 70% positive cells after 1 hour.

FIG. 15: Graphical depiction of differentiation regimen to obtain microglia like cells in neuron-microglia co-cultures. iPSC derived neurons were pre-differentiated for 21 days and could be cryopreserved at this stage of differentiation. To start co-cultures, neurons were thawed at least 1 week prior to seeding of monocytic progenitors. For better visualization of microglia development, movement and morphological properties, GFP positive iPS cells were used for the generation of the blood factories and the monocytic progenitors. For microglia-like differentiation GFP positive monocytic progenitors were plated on top of pre-differentiated neuronal cultures and matured for 2 weeks.

FIGS. 16A-B: Microglia distribution and morphology in co-culture was observed by fluorescence microscopy for GFP expressed in microglia-like cells (FIG. 16A) and neurons labeled with anti-beta-III-tubulin (Tuj) antibody (FIG. 16B). After one week of differentiation monocyte microglia like cells are evenly spread in the co-culture and display a ramified morphology.

FIGS. 17A-H: Cytokine release of macrophages and microglia upon LPS stimulation. One functional property of macrophages and microglia is the capability of releasing cytokines in response to inflammatory stimuli such as LPS. In order to test differences between macrophages, microglia as well as baseline, the baseline cytokine levels of the neural co-culture, the cytokine levels of IL1b (FIG. 17A), IL6 (FIG. 17B), MCP1 (FIG. 17C), IL 10 (FIG. 17D), IL8 (FIG. 17E), IL12p40 (FIG. 17F), MIP1a (FIG. 17G) and TNFa (FIG. 17H) of unstimulated cells and cells stimulated with 100 ng/ml LPS was measured with CBA. Microglia show more release of IL1b, IL6, Il10, TNFa, IL12p40 and MIP1a and less of IL8 compared to macrophages. Neural monocultures showed TNFa, MCP1, MIP1a and IL8 release, indicating contribution of astrocytes to the inflammatory response in co-culture.

FIGS. 18A-D: Phagocytosis is a key functional property of cells of myeloid origin. In order to monitor this process in different culture conditions, e.g. macrophages or microglia, different substrates (such as zymosan, Abeta coated beats or apoptotic cells) labeled with pH sensitive dye pHrodo can be used. These substrates are recognized by myeloid cells, engulfed in endosomes and become fluorescent when the pH drops during lysosomal maturation. Representative images of pHrodo labeled zymosan, which was taken up by either microglia (FIGS. 18A and 18B) or macrophages (FIGS. 18C and 18D).

FIGS. 19A-B: Phagocytotic activity can be used for drug screening employing the pHrodo technology and image based readouts. Interference with the functionality of the cytoskeleton can lead to a decrease in phagocytotic activity (FIG. 19A), while co-incubation or pretreatment with serum (FCS) can lead to a concentration depended increase in zymosan uptake activity (FIG. 19B).

FIGS. 20A-D: For midterm storage of monocytic progenitors and large batch generation, harvested monocytes from blood factories can be cultivated in suspension cultures (“Spinner”). Differentiation of stored monocytic progenitors into macrophages can be initiated at any time point (FIG. 20A). Monocytic progenitors cultivated in suspension cultures (“Spinner”) stay viable for at least 6 weeks (FIG. 20B) and retain their marker profile (FIG. 20C). Macrophages differentiated from monocytic progenitors kept in suspension cultures (“Spinner”) have similar marker expression compared to macrophages differentiated directly after harvesting (FIG. 20D).

FIGS. 21A-B: Macrophages differentiated from monocytic progenitors in suspension cultures (“Spinner”) display indistinguishable functional properties than macrophages differentiated from cells directly after harvesting (“Harvest”), they have similar phagocytotic capacity (FIG. 21A) and migratory capacity (FIG. 21 B).

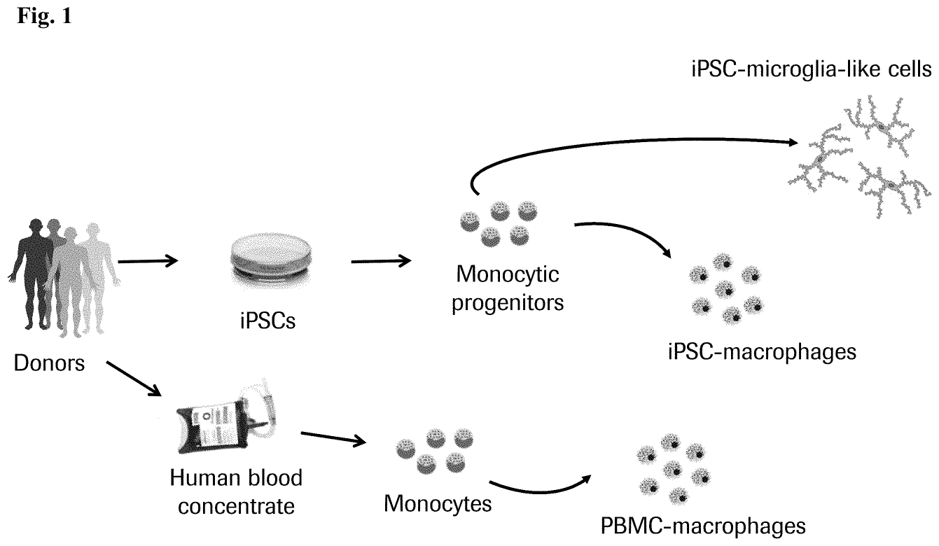

### CITED REFERENCES

Abud E M, Ramirez R N, Martinez E S, et al. (2017) iPSC-Derived Human Microglia-like Cells to Study Neurological Diseases. Neuron 94(2):278-293
Ackermann M, Kempf H, Hetzel M, et al. (2018) Bioreactor-based mass production of human iPSC-derived macrophages enables immunotherapies against bacterial airway infections. Nat Commun 9(1):5088
Biswas S K, Chittezhath M, Shalova I N, Lim J Y (2012) Macrophage polarization and plasticity in health and disease. Immunol Res 53(1-3):11-24
Brownjohn P W, Smith J, Solanki R, et al. (2018) Functional Studies of Missense TREM2 Mutations in Human Stem Cell-Derived Microglia. Stem Cell Reports 10(4):1294-1307
Cassetta L, Pollard J W (2018) Targeting macrophages: therapeutic approaches in cancer. Nat Rev Drug Discov
Davignon J L, Hayder M, Baron M, et al. (2013) Targeting monocytes/macrophages in the treatment of rheumatoid arthritis. Rheumatology (Oxford) 52(4):590-8
Douvaras P, Sun B, Wang M, et al. (2017) Directed Differentiation of Human Pluripotent Stem Cells to Microglia. Stem Cell Reports 8(6):1516-1524
Haenseler W, Sansom S N, Buchrieser J, et al. (2017a) A Highly Efficient Human Pluripotent Stem Cell Microglia Model Displays a Neuronal-Co-culture-Specific Expression Profile and Inflammatory Response. Stem Cell Reports 8(6):1727-1742
Haenseler W, Zambon F, Lee H, et al. (2017b) Excess alpha-synuclein compromises phagocytosis in iPSC-derived macrophages. Sci Rep 7(1):9003
Hong D, Ding J, Li O, et al. (2018) Human-induced pluripotent stem cell-derived macrophages and their immunological function in response to tuberculosis infection. Stem Cell Res Ther 9(1):49
Karlsson K R, Cowley S, Martinez F O, Shaw M, Minger S L, James W (2008) Homogeneous monocytes and macrophages from human embryonic stem cells following coculture-free differentiation in M-CSF and IL-3. Exp Hematol 36(9):1167-75
Mantovani A, Biswas S K, Galdiero M R, Sica A, Locati M (2013) Macrophage plasticity and polarization in tissue repair and remodelling. J Pathol 229(2):176-85
Muffat J, Li Y, Yuan B, et al. (2016) Efficient derivation of microglia-like cells from human pluripotent stem cells. Nat Med 22(11):1358-1367
Peterson K R, Cottam M A, Kennedy A J, Hasty A H (2018) Macrophage-Targeted Therapeutics for Metabolic Disease. Trends Pharmacol Sci 39(6):536-546
Ponzoni M, Pastorino F, Di Paolo D, Perri P, Brignole C (2018) Targeting Macrophages as a Potential Therapeutic Intervention: Impact on Inflammatory Diseases and Cancer. Int J Mol Sci 19(7)
Senju S, Haruta M, Matsumura K, et al. (2011) Generation of dendritic cells and macrophages from human induced pluripotent stem cells aiming at cell therapy. Gene Ther 18(9):874-83
Sica A, Larghi P, Mancino A, et al. (2008) Macrophage polarization in tumour progression. Semin Cancer Biol 18(5):349-55
Takamatsu K, Ikeda T, Haruta M, et al. (2014) Degradation of amyloid beta by human induced pluripotent stem cell-derived macrophages expressing Neprilysin-2. Stem Cell Res 13(3 Pt A):442-53
Tiwari R L, Singh V, Barthwal M K (2008) Macrophages: an elusive yet emerging therapeutic target of atherosclerosis. Med Res Rev 28(4):483-544
van Wilgenburg B, Browne C, Vowles J, Cowley S A (2013) Efficient, long term production of monocyte-derived macrophages from human pluripotent stem cells under partly-defined and fully-defined conditions. PLoS One 8(8):e71098
Wynn T A, Chawla A, Pollard J W (2013) Macrophage biology in development, homeostasis and disease. Nature 496(7446):445-55

### DETAILED DESCRIPTION

As used herein, the term “defined medium” or “chemically defined medium” refers to a cell culture medium in which all individual constituents and their respective concentrations are known. Defined media may contain recombinant and chemically defined constituents.

As used herein the term “differentiating”, “differentiation” and “differentiate” refers to one or more steps to convert a less-differentiated cell into a somatic cell, for example to convert a pluripotent stem cell into a monocyte or to convert a monocyte into a macrophage. Differentiation is achieved by methods known in the art and also described herein.

As used herein, “monocytic progenitor cells” are cells that express the specific surface markers CD14 (Cluster of Differentiation 14, also known as Myeloid cell-specific leucine-rich glycoprotein, official symbol CD14), CD11b (Cluster of Differentiation 11B, also known as Integrin alpha M (ITGAM), macrophage-1 antigen (Mac-1) and complement receptor 3 (CR3/CR3A), official symbol ITGAM), CD68 (Cluster of Differentiation 68, also known as GP110, Macrosialin, scavenger receptor class D member 1 (SCARD1) and LAMP4, official symbol CD68), are in suspension and possess the ability to give rise to adherent macrophages and microglia.

As used herein, “macrophages” are cells that express the specific marker CD14 (Cluster of Differentiation 14, also known as Myeloid cell-specific leucine-rich glycoprotein, official symbol CD14), CD11b (Cluster of Differentiation 11B, also known as Integrin alpha M (ITGAM), macrophage-1 antigen (Mac-1) and complement receptor 3 (CR3/CR3A), official symbol ITGAM), CD68 (Cluster of Differentiation 68, also known as GP110, Macrosialin, scavenger receptor class D member 1 (SCARD1) and LAMP4, official symbol CD68), are adherent, are able to phagocytose different substrates, respond to various inflammatory stimuli and can be polarized by the presence of distinct cytokines (e.g. IL-4 and INFg).

As used herein, “microglia”, are cells that express the specific marker CD14 (Cluster of Differentiation 14, also known as Myeloid cell-specific leucine-rich glycoprotein, official symbol CD14), CD11b (Cluster of Differentiation 11B, also known as Integrin alpha M (ITGAM), macrophage-1 antigen (Mac-1) and complement receptor 3 (CR3/CR3A), official symbol ITGAM), CD68 (Cluster of Differentiation 68, also known as GP110, Macrosialin, scavenger receptor class D member 1 (SCARD1) and LAMP4, official symbol CD68), IBA 1 (ionized calcium-binding adaptor molecule 1, also known as Allograft inflammatory factor 1AIF1, official symbol AIF1), have a ramified morphology, are able to phagocytose different substrates, respond to various inflammatory stimuli and express at least one further marker protein for example TMEM119 (transmembrane protein 119, also known as Osteoblast induction factor (OBIF), official symbol TMEM119), P2RY12 (P2Y purinoceptor 12, also known as ADP-glucose receptor, official symbol P2RY12) or PROS1(protein S, also known as PSA; PROS; PS21; PS22; PS23; PS24; PS25; THPH5; THPH6, official symbol PROS1) and/or are of ramified morphology.

A “mesoderm induction medium” as used herein refers to any medium, preferably a chemically defined medium, useful for the induction of mesoderm in pluripotent stem cells. One example of such medium is a defined medium, e.g. MTeSR1 medium, supplemented with human recombinant bone morphogenic protein-4 (BMP4), human vascular endothelial growth factor (VEGF) and human stem cell factor (SCF). Suitable markers to determine mesoderm induction are MIXL, EOMES and T-brachyury.

A “myeloid maturation medium” as used herein refers to a medium, preferably a chemically defined medium, useful for the maturation of cells along the myeloid lineage. One example of such medium is a defined medium, e.g. XVIVO15 medium, supplemented with macrophage colony-stimulating factor (M-SCF) and interleukin 3 (IL-3). Suitable marker to determine maturation along the myeloid lineage are CD14, ITGAM and/or CD68.

A “macrophage differentiation medium” as used herein refers to any medium, preferably a chemically defined medium, useful for the differentiation of monocytic progenitor cells into macrophages. One example of such medium is a defined medium, e.g.)(VIVO'S medium, supplemented with macrophage colony-stimulating factor (M-CSF). Suitable macrophage markers to identify macrophages are CD14, ITGAM and/or CD68 as well as adherence to cell culture substrates, phagocytosis, response to various inflammatory stimuli and polarization upon treatment with e.g., IL-4 and/or INFg.

As used herein, the term “growth factor” means a biologically active polypeptide or a small molecule compound which causes cell proliferation, and includes both growth factors and their analogs.

“High-throughput screening” as used herein shall be understood to signify that a large number of different disease model conditions and/or chemical compounds are analyzed and compared in parallel. Typically, such high-throughput screening (assays) are performed in multi-well microtiter plates, e.g., in a 96 well plate or a 384 well plate or plates with 1536 or 3456 wells.

A “large scale cell culture” as used herein refers to a cell culture (system) wherein a large amount of cells are confined under conditions (e.g., medium supply, gas exchange, available surface area) to maintain viability of the cells wherein the amount of cells is suitable for high-throughput screening (assays). In particular embodiments, a large scale cell culture containment (e.g., vessel, container, flask) comprises more than 10⁶, 10⁷, 10⁸, 10⁹, 10¹⁰, 10¹¹, 10¹² cells. In one embodiment, the large scale cell culture comprises one single cell culture containment. In another embodiment, the large scale cell culture comprises an assembly of multiple cell culture containments. In further embodiment, the large scale cell culture (containment) comprises a cell culture area of at least 100 cm², 500 cm², 1'000 cm², 2'000 cm², 5'000 cm², 10'000 cm². In one embodiment, the large scale cell culture (system) is inoculated with at least 1, 2, 3, 4, 5 embryoid bodies per cm² corresponding to a starting cell number at day 1 of at least 10⁵, 10⁶, 10⁷, 10⁸, 10⁹ cells. In one embodiment, one embryoid body (corresponding to about 13'000 cells) is seeded per cm² of cell culture area.

A “monolayer of cells” as used herein means that the cells are attached to an adhesive substrate (e.g., cell culture support) substantially as one single layer of cells, as opposed to non-confluent single cells and opposed to a plurality of cells forming (multiple) three dimensional layered or non-layered formations (e.g., embryoid bodies) attached to or non-attached to the adhesive substrate.

“Pluripotency medium” as used herein refers to any chemically defined medium useful for the attachment of pluripotent stem cells as single cells in a monolayer while maintaining their pluripotency. Useful pluripotency media are well known in the art and also described herein. In particular embodiments as described herein, the pluripotency medium contains at least one of the following growth factors: basic fibroblast growth factor (bFGF, also depicted as Fibroblast Growth Factor 2, FGF2) and transforming growth factor β (TGFβ).

As used herein, the term “reprogramming” refers to one or more steps needed to convert a somatic cell to a less-differentiated cell, for example for converting a fibroblast cell, adipocytes, keratinocytes or leucocyte into a pluripotent stem cell. “Reprogrammed” cells refer to cells derived by reprogramming somatic cells as described herein.

The term “small molecule”, or “small compound”, or “small molecule compound” as used herein, refers to organic or inorganic molecules either synthesized or found in nature, generally having a molecular weight less than 10,000 grams per mole, optionally less than 5,000 grams per mole, and optionally less than 2,000 grams per mole.

The term “somatic cell” as used herein refers to any cell forming the body of an organism that are not germline cells (e.g., sperm and ova, the cells from which they are made (gametocytes)) and undifferentiated stem cells.

The term “stem cell” as used herein refers to a cell that has the ability for self-renewal. An “undifferentiated stem cell” as used herein refers to a stem cell that has the ability to differentiate into a diverse range of cell types. As used herein, “pluripotent stem cells” refers to a stem cell that can give rise to cells of multiple cell types. Pluripotent stem cells (PSCs) include human embryonic stem cells (hESCs) and human induced pluripotent stem cells (hiPSCs). Human induced pluripotent stem cells can be derived from reprogrammed somatic cells, e.g. by transduction of four defined factors (Sox2, Oct4, Klf4, c-Myc) by methods known in the art and further described herein. Said human somatic cells can be obtained from a healthy individual or from a patient. These donor cells can be obtained from any suitable source. Preferred herein are sources that allow isolation of donor cells without invasive procedures on the human body, for example human skin cells, blood cells or cells obtainable from urine samples.

The term “suspension culture” as used herein refers to a cell culture system wherein the cells (single cells or aggregates of cells, e.g., embryoid bodies) substantially do not or only minimally attach to (a) surface(s) of (a) cell culture containment(s) used to incubate the cells. In suspension culture, cells or cell aggregates float with minimal or no contact to a cell culture containment surface (e.g. a tissue culture support of a flask). Minimally attached cells or cell aggregates of suspension cultures can be readily detached by use of weak or moderate physical force, such as e.g., mild shaking, tapping or horizontal movement of the cell culture.

The term “adherent cell culture” as used herein refers to a cell culture system wherein the cells, as in contrast to suspension cultures, attach to (a) surface(s) of (a) cell culture containment(s) used to incubate the cells. Minimally attached cells or cell aggregates of suspension cultures which can be readily detached by use of weak or moderate physical force as herein described are not considered adherent cell cultures.

Although human cells are preferred, the methods as herein described are also applicable to non-human cells, such as primate, rodent (e.g. rat, mouse, rabbit) and dog cells.

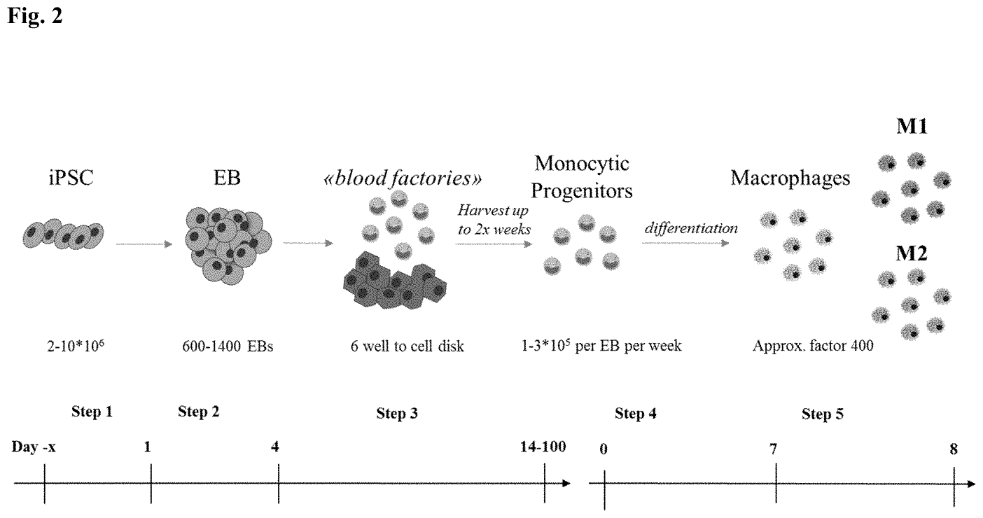

Herein provided is a method for producing monocytic progenitor cells. Before the present invention several technical issues limited the use of monocytes and macrophages in drug discovery. In order to guarantee project delivery in time, factors such as cell number, scalability, reproducibility and phenotypic relevance are essential. The present inventors modified a published protocol (van Wilgenburg et al. 2013) and could increase the yield and reproducibility, while decreasing the differentiation time. In a preferred embodiment, embryoid bodies (EB's) are generated from induced pluripotent stem cells (iPSCs) plated on a cell culture support coated with laminin. These EB's resemble the early embryo formation and initiate the formation of the three germ layers (primitive streak). The EB's are then pre-differentiated by contacting the cells with a defined medium comprising BMP4 to direct cell commitment to the mesodermal lineage. Once formed and pre-differentiated the EB's are plated and further differentiated along the myeloid lineage to form blood factories, which produce and release monocytic-progenitors in the supernatant (FIG. 2). The blood factories can be maintained for more than 100 days and monocytic-progenitors can be harvested from the culture supernatant (up to twice a week). After harvesting, these progenitors can be differentiated to un-polarized macrophages within one week, or further polarized by specific cytokine addition promoting either pro- or anti-inflammatory subtypes. By increasing the scalability of the blood factories from 10 to 1000 cm² culture area the present invention achieves cell harvest and handling times that fit the needs of drug discovery and development project related work as well as medium sized drug screen programs. In another aspect a new co-culture setting for generation of microglia-like cells was established.

Generation of Monocytic Progenitor Cells

Pluripotent stem cells have self-renewal character and can be differentiated in all major cell types of the adult mammalian body. Pluripotent stem cells can be produced in large quantities under standardized cell culture conditions. Accordingly, in a preferred embodiment, the monocytic progenitor cells are generated, i.e. differentiated, from pluripotent stem cells. In one embodiment, the monocytic progenitor cells are generated, i.e. differentiated from embryonic stem cells. In a preferred embodiment, the monocytic progenitor cells are generated, i.e. differentiated, from induced pluripotent stem cells (iPSCs). In one embodiment the iPSCs are generated from reprogrammed somatic cells. Reprogramming of somatic cells to iPSCs can be achieved by introducing specific genes involved in the maintenance of iPSC properties. Genes suitable for reprogramming of somatic cells to IPSCs include, but are not limited to Oct4, Sox2, Klf4 and C-Myc and combinations thereof. In one embodiment the genes for reprogramming are Oct4, Sox2, Klf4 and C-Myc.

Internal organs, skin, bones, blood and connective tissue are all made up of somatic cells. Somatic cells used to generate iPSCs include but are not limited to fibroblast cells, adipocytes and keratinocytes and can be obtained from skin biopsy. Other suitable somatic cells are leucocytes, erythroblasts cells obtained from blood samples or epithelial cells or other cells obtained from blood or urine samples and reprogrammed to iPSCs by the methods known in the art and as described herein. The somatic cells can be obtained from a healthy individual or from a diseased individual. In one embodiment, the somatic cells are derived from a subject (e.g., a human subject) suffering from a disease. In one embodiment, the disease is associated with either chronic inflammation (e.g. Inflammatory bowel disease), primary or acquired immune deficiency (e.g. bare lymphocyte syndrome) or neurodegenerative diseases (e.g. Multiple Sclerosis, Alzheimer or Parkinson's Disease). The genes for reprogramming as described herein are introduced into somatic cells by methods known in the art, either by delivery into the cell via reprogramming vectors or by activation of said genes via small molecules. Methods for reprogramming comprise, inter alia, retroviruses, lentiviruses, adenoviruses, plasmids and transposons, microRNAs, small molecules, modified RNAs messenger RNAs and recombinant proteins. In one embodiment, a lentivirus is used for the delivery of genes as described herein. In another embodiment, Oct4, Sox2, Klf4 and C-Myc are delivered to the somatic cells using Sendai virus particles. In addition, the somatic cells can be cultured in the presence of at least one small molecule. In one embodiment, said small molecule comprises an inhibitor of the Rho-associated coiled-coil forming protein serine/threonine kinase (ROCK) family of protein kinases. Non-limiting examples of ROCK inhibitors comprise fasudil (1-(5-Isoquinolinesulfonyl) homopiperazine), Thiazovivin (N-Benzyl-2-(pyrimidin-4-ylamino) thiazole-4-carboxamide) and Y-27632 ((+)-(R)-trans-4-(1-aminoethyl)-N-(4-pyridyl) cyclo-hexanecarboxamide dihydrochloride).

Providing a defined monolayer of pluripotent stem cells is preferred for reproducibility and efficiency of the resulting cultures. The present inventors surprisingly found that the substitution by using laminin coating substrate in the stem cell maintenance culture decreased the differentiation time of blood factories and increased the throughput of the cell cultures. In one embodiment, monolayers of pluripotent stem cells can be produced by enzymatically dissociating the cells into single cells and plating them onto an adhesive substrate, e.g. on cell culture containments (e.g., flasks) coated with the laminin substrate. In a preferred embodiment, the adhesive substrate (coating) is laminin. In one embodiment, the laminin comprises laminin subunit alpha-4. In one embodiment, the laminin comprises laminin subunit alpha-5. In one embodiment, the laminin comprises laminin subunit beta-1. In one embodiment, the laminin comprises laminin subunit beta-2. In one embodiment, the laminin comprises laminin subunit gamma-1. In one embodiment, the laminin comprises laminin subunits alpha-4, beta-1 and gamma-1 (Laminin-411). In one embodiment, the laminin comprises laminin subunits alpha-5, beta-1 and gamma-1 (Laminin-511). In a preferred embodiment, the laminin comprises laminin subunits alpha-5, beta-2 and gamma-1 (Laminin-521, e.g. BioLamina rhLaminin-521).

Examples of enzymes suitable for the dissociation into single cells include Accutase (Invitrogen), Trypsin (Invitrogen), TrypLe Express (Invitrogen). In one embodiment, 20'000 to 60'000 cells per cm² are plated on the adhesive substrate. The medium used herein is a pluripotency medium which facilitates the attachment and growth of the pluripotent stem cells as single cells in a monolayer. In one embodiment, the pluripotency medium is a serum free medium supplemented with a small molecule inhibitor of the Rho-associated coiled-coil forming protein serine/threonine kinase (ROCK) family of protein kinases (herein referred to as ROCK kinase inhibitor).

Thus, in one embodiment, the method described herein comprises providing a monolayer of pluripotent stem cells in a pluripotency medium on a laminin substrate, wherein said pluripotency medium is a serum free medium supplemented with a ROCK kinase inhibitor.

Examples of serum-free media suitable for the attachment of the pluripotent stem cells to the substrate are mTeSR1 or TeSR2 from Stem Cell Technologies, Primate ES/iPS cell medium from ReproCELL, PluriSTEM from Milipore, StemMACS iPS-Brew frp Milenyi Biotec and StemPro hESC SFM from Invitrogen, X-VIVO from Lonza. Examples of ROCK kinase inhibitor useful herein are Fasudil (1-(5-Isoquinolinesulfonyl)homopiperazine), Thiazovivin (N-Benzyl-2-(pyrimidin-4-ylamino)thiazole-4-carboxamide) and Y27632 ((+)-(R)-trans-4-(1-aminoethyl)-N-(4-pyridyl) cyclo-hexanecarboxamide dihydrochloride, e.g. Catalogue Number: 1254 from Tocris bioscience). In one embodiment, the pluripotency medium is a serum free medium supplemented with about 2-20 μM Y27632, preferably about 5-10 μM Y27632. In another embodiment the pluripotency medium is a serum free medium supplemented with about 2-20 μM Fasudil. In another embodiment the pluripotency medium is a serum free medium supplemented with about 0.2-10 μM Thiazovivin.

In one embodiment the method described herein comprises providing a monolayer of pluripotent stem cells in a pluripotency medium on a laminin substrate and growing said monolayer in the pluripotency medium for at least one day (24 hours). In another embodiment the method described herein comprises providing a monolayer of pluripotent stem cells in a pluripotency medium and growing said monolayer in the pluripotency medium for 18 hours to 30 hours, preferably for 23 to 25 hours. In further embodiments method described herein comprises providing a monolayer of pluripotent stem cells in a pluripotency medium on a laminin substrate and growing said monolayer in the pluripotency medium for at least 2, 3, 4, 5, 6, 7, 8, 9, 10 days or more than 10 days.

In another embodiment the method described herein comprises providing a monolayer of pluripotent stem cells in a pluripotency medium on a laminin substrate, wherein said pluripotency medium is mTesR1 medium, and growing said monolayer in the pluripotency medium for one day (24 hours). In another embodiment the method described herein comprises providing a monolayer of pluripotent stem cells in a pluripotency medium on a laminin substrate, wherein said pluripotency medium is mTesR1, and growing said monolayer in the pluripotency medium for 18 hours to 30 hours, preferably for 23 to 25 hours.

In a next step b), the pluripotent stem cells are harvested and transferred to a suspension culture. In one embodiment, the pluripotent stem cells are contacted with a mesoderm induction medium. In one embodiment, the mesoderm induction medium comprises recombinant bone morphogenic protein-4 (BMP4). In one embodiment, the mesoderm induction medium is a serum free medium supplemented with about 10-100 ng/ml BMP4 (e.g. hBMP4), preferably about 50 ng/ml BMP4.

In a further embodiment, the mesoderm induction medium additionally comprises vascular endothelial growth factor (VEGF). In one embodiment, the mesoderm induction medium is a serum free medium supplemented with about 10-100 ng/ml VEGF (e.g. hVEGF), preferably about 50 ng/ml VEGF.

In a further embodiment, the mesoderm induction medium additionally comprises stem cell factor (SCF). In one embodiment, the mesoderm induction medium is a serum free medium supplemented with about 5-50 ng/ml SCF (e.g. hSCF), preferably about 20 ng/ml SCF.

In a preferred embodiment, the mesoderm induction medium comprises BMP4, VEGF and SCF, in particular about 10-100 ng/ml BMP4, about 10-100 ng/ml VEGF and about 5-50 ng/ml SCF. In a preferred embodiment, the mesoderm induction medium comprises about 50 ng/ml BMP4, about 50 ng/ml VEGF and about 20 ng/ml SCF

In one embodiment the pluripotent stem cells are contacted with the mesoderm induction medium for at least about one day (24 hours). In further embodiments the pluripotent stem cells are contacted with the mesoderm induction medium for about 2, 3, 4, 5, 6, 7, 8, 9, 10 days or more than about 10 days. In one embodiment the pluripotent stem cells are contacted with the mesoderm induction medium for about 24 hours to about 72 hours, preferably for about 36 to about 60 hours.

In one embodiment, the cells are plated in step c) on a cell culture support suitable for attachment of the cells after mesoderm induction. In a preferred embodiment, the cells are plated on a cell culture support coated with a basement membrane biomaterial, such as e.g., Matrigel, Cultrex BME, Geltrex Matrix. In one embodiment the basement membrane biomaterial comprises laminin, collagen IV, heparin sulfate proteoglycans and entactin/nidogen-1,2. In a preferred embodiment, the cells are plated on a cell culture support coated with Matrigel.

In one embodiment, the cells are plated in step c) in a large scale cell culture container. In particular embodiments, more than 10⁶, 10⁷, 10⁸, 10⁹, 10¹⁰, 10¹¹, 10¹² cells are seeded into one individual the large scale cell culture containment. In one embodiment, the large scale cell culture comprises one single cell culture containment. In another embodiment, the large scale cell culture comprises an assembly of multiple cell culture containments. In further embodiment, the large scale cell culture (containment) comprises a cell culture area of at least 100 cm², 500 cm², 1'000 cm², 2'000 cm², 5'000 cm², 10'000 cm². In one embodiment, the large scale cell culture (system) is inoculated with at least 10⁶, 10⁷, 10⁸, 10⁹, 10¹⁰, 10¹¹, 10¹² cells.

In a next step, the cells in the large scale cell cultures are further differentiated along the myeloid lineage. In one embodiment, the plated cells are contacted in step c) with a myeloid maturation medium. Suitable myeloid maturation media are known in the art and also described herein. In one embodiment, the myeloid maturation medium comprises interleukin 3 (IL-3). In one embodiment, the myeloid maturation medium is a serum free medium supplemented with about 1-50 ng/ml IL-3 (e.g. hIL-3), preferably about 25 ng/ml IL-3. In one embodiment the cells are contacted with the myeloid maturation medium for about 4 days (about 96 hours). In further embodiments the cells are contacted with the myeloid maturation medium for about 2, 3, 4, 5, 6, 7, 8, 9, 10 days or more than about 10 days. In one embodiment the cells are contacted with the myeloid maturation medium for about 72 hours to about 120 hours, preferably for about 84 to about 108 hours. During the step of myeloid maturation, the large scale cell cultures begin to produce monocytic progenitor cells. Monocytic progenitor cells can be harvested from the adherent cells culture after myeloid maturation by collecting the supernatant of the cell culture. In one embodiment, the large scale cell cultures of step c) according to the present invention are capable of producing monocytic progenitor cells for more than about 10, 15, 20, 25, 30, 40, 50, 60, 70, 80, 90 or 100 days. In one embodiment, the large scale cultures of step c) are capable of producing at least about 100'000 monocytic progenitor cells per cm² of cell culture area per week.

Differentiation of Monocytic Progenitor Cells into Macrophages

Monocytic progenitor cells can be differentiated into macrophages by methods known in the art and also as herein described. In one embodiment, monocytic progenitor cells are contacted with a macrophage differentiation medium. In one embodiment, the cells are contacted with a macrophage differentiation medium for about 1-10 days, 4-8 days, 2 days, 3 days, 4 days, 5 days, 6 days, 7 days, 8 days, 9 days or for more than about 10 days. In one embodiment, the macrophage differentiation medium comprises macrophage colony-stimulating factor (M-CSF). In one embodiment, the macrophage differentiation medium is a serum free medium supplemented with 10-200 ng/ml M-CSF (e.g. hM-CSF), preferably 100 ng/ml M-CSF. In a preferred embodiment, the cells are contacted with the macrophage differentiation medium for about 6 days. In one embodiment, the cells are plated onto a non-coated tissue culture support prior to or concomitant with contacting the cells with the macrophage differentiation medium. In one embodiment, the macrophages are re-plated onto a non-coated tissue culture support. In one embodiment, the macrophages are re-plated in a high-throughput plat format. In one embodiment, macrophages are re-plated in 24-well plate format, in 96-well plate format, or 384-well plate format.

Differentiation of Monocytic Progenitor Cells into Microglia

Monocytic progenitor cells can be differentiated into microglia by methods known in the art and also as herein described. In one embodiment, monocytic progenitor cells are contacted with neurons. In one embodiment, the neurons are generated using the methods as described in WO2017081250. In some embodiment, the neurons are differentiated for (at least) about 1 week, 2 weeks, 3 weeks, 4 weeks, 5 weeks, 6 weeks, 7 weeks, 8 weeks, 9 weeks, 10 weeks. In a preferred embodiment, the neurons are differentiated for about 2-5 weeks. In some embodiments, the cells are contacted with neurons for about 2 days, 3 days, 4 days, 5 days, 6 days, 7 days, 8 days, 9 days or for more than about 10 days. In some embodiment, the cells are contacted with neurons for about 5-20 days or about 10-18 days. In one embodiment, the cells are co-cultured with neurons in a co-culture differentiation medium. In one embodiment, the co-culture differentiation medium comprises granulocyte macrophage colony-stimulating factor (GM-CSF) and/or interleukin 34 (IL-34). In one embodiment, co-culture differentiation medium is a serum free medium supplemented with 10-200 ng/ml GM-CSF (e.g. hGM-CSF), preferably 100 ng/ml GM-CSF. In one embodiment, co-culture differentiation medium is a serum free medium supplemented with 1-500 ng/ml IL-34 (e.g. hIL-34), preferably 100 ng/ml IL-34. In a preferred embodiment, the cells are contacted with neurons and the co-culture differentiation medium for about 14 days in a serum free medium supplemented with 10-200 ng/ml GM-CSF (e.g. hGM-CSF) and 1-500 ng IL-34, preferably 100 ng/ml GM-CSF and 100 ng/ml IL-34.

### Exemplary Embodiments

1. A method for producing monocytic progenitor cells, the method comprising the step of:
a) plating pluripotent stem cells in a pluripotency medium on a cell culture support coated with laminin;
b) harvesting the pluripotent stem cells and contacting the pluripotent stem cells with a mesoderm induction medium in suspension culture;
c) plating the cells on a cell culture support suitable for attachment of the cells; and
d) harvesting the monocytic progenitor cells from suspension.
   2. The method of embodiment 1, wherein the cells in step a) are cultured for at least about 1 day, 2 days, 3 days, 4 days, 5 days, 6 days, 7 days, 8 days, 9 days or 10 days on the cell culture support coated with laminin, in particular for at least about 1 day.
   3. The method of embodiment 1 or 2, wherein the laminin in step a) comprises laminin subunit alpha-5, in particular wherein the laminin in step a) comprises laminin subunits alpha-5, beta-2 and gamma 1.
   4. The method of any one of embodiments 1 to 3, wherein the mesoderm induction medium is a chemically defined medium comprising recombinant bone morphogenic protein-4 (BMP4).
   5. The method of embodiment 4, wherein the medium comprises about 10-100 ng/ml BMP4, preferably about 50 ng/ml BMP4.
   6. The method of any one of embodiments 4 or 5, wherein the mesoderm induction medium additionally comprises vascular endothelial growth factor (VEGF).
   7. The method of embodiment 6, wherein the mesoderm induction medium comprises about 10-100 ng/ml VEGF, preferably about 50 ng/ml VEGF.
   8. The method of any one of embodiments 4 to 7, wherein the mesoderm induction medium additionally comprises stem cell factor (SCF).
   9. The method of embodiment 8, wherein the mesoderm induction medium comprises about 5-50 ng/ml SCF, preferably about 20 ng/ml SCF.
   10. The method of any one of embodiments, 1 to 9, wherein the cells are contacted with the mesoderm induction medium for about 1-10 days, 2-6 days, 2 days, 3 days, 4 days, 5 days, 6 days, 7 days, 8 days, 9 days.
   11. The method of any one of embodiments 1 to 10, wherein the cells are contacted with the mesoderm induction medium for about 4 days.
   12. The method of any one of embodiments 1 to 11, wherein the cells in step b) form embryoid bodies (EBs).
   13. The method of any one of embodiments 1 to 12, wherein the cell culture support in step c) is coated with a basement membrane biomaterial.
   14. The method of embodiment 13, wherein the basement membrane biomaterial comprises laminin, collagen IV, heparin sulfate proteoglycans and entactin/nidogen-1,2.
   15. The method of any one of embodiments 1 to 14, wherein the cells in step c) are contacted with a myeloid maturation medium.
   16. The method of any one of embodiments 1 to 15, wherein the myeloid maturation medium comprises macrophage colony-stimulating factor (M-CSF).
   17. The method of embodiment 16, wherein the myeloid maturation medium comprises about 20-200 ng/ml M-CSF, preferably about 100 ng/ml M-CSF.
   18. The method of any one of embodiments 15 to 17, wherein the myeloid maturation medium additionally comprises IL-3.
   19. The method of embodiment 18, wherein the medium comprises about 1-50 ng/ml IL-3, preferably about 25 ng/ml IL-3.
   20. The method of any one of embodiments 15 to 19, wherein the cells are contacted with the myeloid maturation medium for about 1-10 days, 2-6 days, 2 days, 3 days, 4 days, 5 days, 6 days, 7 days, 8 days, 9 days.
   21. The method of any one of embodiments 15 to 20, wherein the cells are contacted with the myeloid maturation medium for about 4 days.
   22. The method of any one of embodiments 1 to 21, wherein the monocytic progenitor cells are harvested in step d) by collecting the supernatant of the cell culture.
   23. The method of any one of embodiments 1 to 22, wherein the monocytic progenitor cells are harvested in batches by collecting the supernatant of the cell culture in step d).
   24. The method of any one of embodiments 1 to 23, wherein the monocytic progenitor cells are harvested in batches at regular intervals; in particular every day, every other day, every 3, every 4, every 5 or every 6 days.
   25. The method of any one of embodiments 1 to 24, wherein the monocytic progenitor cells are harvested continuously.
   26. The method of any one of embodiments 1 to 25, wherein the monocytic progenitor cells are harvested continuously by removing supernatant from the cell culture in step d) and, optionally replacing the removed supernatant with fresh medium.
   27. The method of any one of embodiments 1 to 26, further comprising step e) differentiating the harvested monocytic progenitor cells into macrophages.
   28. The method of embodiment 27, wherein the cells in step e) are contacted with a macrophage differentiation medium.
   29. The method of embodiment 27, wherein the macrophage differentiation medium comprises macrophage colony-stimulating factor (M-CSF).
   30. The method of embodiment 28 or 29, wherein the macrophage differentiation medium comprises about 10-200 ng/ml M-CSF, preferably about 100 ng/ml M-CSF.
   31. The method of any one of embodiments 28 to 30, wherein the cells are contacted with the macrophage differentiation medium for about 1-10 days, 4-8 days, 2 days, 3 days, 4 days, 5 days, 6 days, 7 days, 8 days, 9 days.
   32. The method of any one of embodiments 28 to 31, wherein the cells are contacted with the macrophage differentiation medium for about 6 days.
   33. The method of any one of embodiments 28 to 32, wherein the cells in step e) are plated onto a non-coated tissue culture support.
   34. The method of any one of embodiments 28 to 33, wherein the macrophages are re-plated onto a non-coated tissue culture support.
   35. The method of any one of embodiments 28 to 34, wherein the macrophages are re-plated in 24-well plate format, in 96-well plate format, or 384-well plate format.
   36. The method of any one of embodiments 1 to 26, further comprising step e) differentiating the monocytic progenitor cells into microglia.
   36. The method of embodiment 35, wherein the monocytic progenitor steps in step e) are co-cultured with neuronal cells 37. The method of embodiment 35 or 36, wherein the cells in step e) are contacted with a co-culture differentiation medium.
   38. The method of embodiment 37, wherein the co-culture differentiation medium comprises granulocyte macrophage colony-stimulating factor (GM-CSF) and/or interleukin 34 (IL-34).
   39. The method of embodiment 38, wherein the co-culture differentiation medium comprises about 10-200 ng/ml GM-CSF, preferably about 100 ng/ml GM-CSF.
   40. The method of embodiment 38, or 39 wherein co-culture differentiation medium medium comprises about 1-500 ng/ml IL-34 (e.g. hIL-34), preferably about 100 ng/ml IL-34.
   41. The method of any one of embodiments 38 to 40, wherein the cells are contacted with the co-culture differentiation medium for about 1-28 days, 7-21 days, 8 days, 9 days, 10 days, 11 days, 12 days, 13 days, 14 days, 15 days.
   42. The method of any one of embodiments 38 to 41, wherein the cells are contacted with the co-culture differentiation medium for about 14 days.
   43. The method of any one of embodiments 36 to 42, wherein the neuronal cells are derived from pluripotent stem cells.
   44. The method of any one of embodiments 36 to 43, wherein the neuronal cells are produced according to the methods for producing standardized cell cultures of uniformly distributed differentiated NCs as described in WO/2017/081250.
   45. The method of any one of embodiments 1 to 44, wherein the pluripotent stem cells are mammalian cells, in particular human cells.
   46. The method of any one of embodiments 1 to 45, wherein the pluripotent stem cells are embryonic stem cells (ESCs).
   47. The method of any one of embodiments 1 to 45, wherein the pluripotent stem cells induced pluripotent stem cells (IPSCs).
   48. An adherent large scale cell culture for producing monocytic progenitor cells, wherein the adherent cell culture is capable of producing at least about 100'000 monocytic progenitor cells per cm² of cell culture area per week.
   49. The adherent large scale cell culture of embodiment 48 produced by the method of any one of claims 1 to 26 steps a) to c).
   49. The invention as hereinbefore described.

### EXAMPLES

The following are non-limiting examples of compositions and methods of the invention. It is understood that various other embodiments may be practiced, given the general description provided above.

Materials and Methods

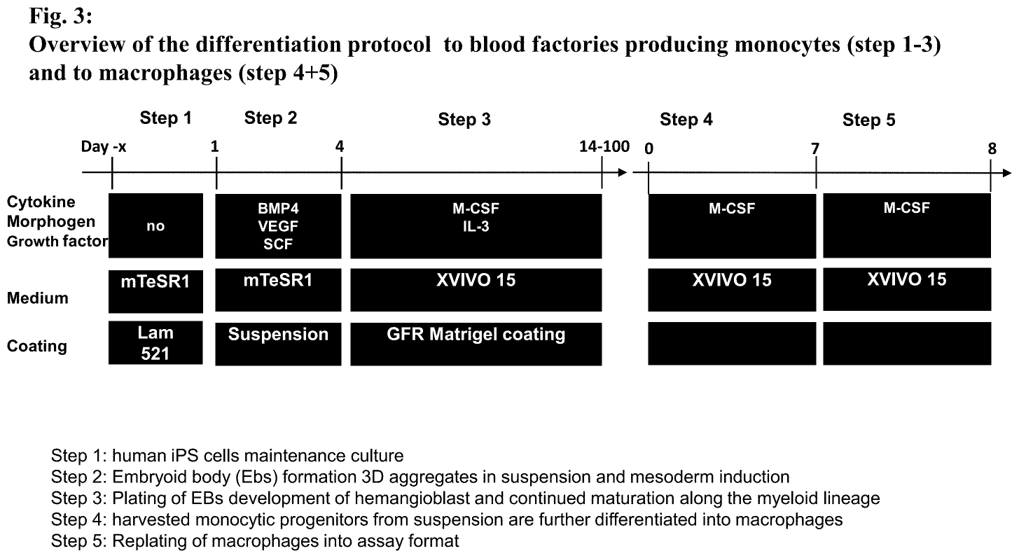

In order to generate macrophages from human induced pluripotent stem cells, we adopted the published protocol of (van Wilgenburg et al. 2013). This resulted in a multistep protocol depicted in FIG. 3. The protocol is structured in five steps: iPSC maintenance (Step 1), EB formation (Step2), Plating of EBs (Step3), Macrophage differentiation (Step4) and Macrophage polarization (Step5).

iPSC Maintenance in Feeder Free Conditions

Culture dishes (Corning) were coated with 12.5 ug/ml rhLaminin-521 (BioLamina) in PBS containing calcium and magnesium for at least 2 hours prior to use. hiPS cells were seeded and cultured in mTesR1 medium (StemCell Technologies) at 37° C. with 5% CO² and medium was changed daily. Cells were passaged at 90% confluency. Therefore media was removed, cells were washed 1× with PBS and detached with accutase for 2 to 5 minutes at 37° C. After removal of accutase by centrifugation cells were either used for maintenance or start of differentiation.

EBs Formation and Mesoderm Induction

To obtain uniformed EBs, iPS cells were plated into Aggrewell 800 (StemCell Technologies) plates. Therefore, 2 ml mTesR1, supplemented with 10 μM ROCK inhibitor (Y27632, Callbiochem) and containing 4*10⁶ iPS single cells, was added to each Aggrewell and centrifuged for 3 minutes at 100 g to assure an even and fast distribution of the iPS cells to the aggrewell microwells. The next day mesoderm induction was started by exchange of 75% (replacing twice 1 ml of the 2 ml in each well) of the mTeSR1 media with fresh mTeSR1 media supplemented with 50 ng/ml hBMP4, 50 ng/ml hVEGF and 20 ng/ml hSCF. For further differentiation this was repeated the following 2 days.

Plating of EBs and Continued Maturation Along the Myeloid Lineage

At day 4 of differentiation EBs were harvested by gently dislodging the EBs by rinsing the aggrewells with PBS. EBs were collected in a 40 μm strainer and transferred to factory media, consisting of XVIVO15 media (Lonza) supplemented with 2 mM Glutamax, 1% Penicillin/Streptomycin, 50 ug/ml Mercaptoethanol, M-CSF (20-200 ng/ml) and IL3 (1-50 ng/ml). EBs were plated with a density of 0.8-1.5 EBs/cm² on cell culture vessels of desired surface areas (2-2000 cm²) pre-coated for 1 h at RT with growth factor reduced Matrigel (354230 Corning) diluted in cold DMEM F12 1:1 1× Glutamax Gibco 31331-028). In order to allow adherence of EBs, EBs were evenly distributed by slow movements and culture vessels were placed immediately at 37° C. with 5% CO² without any further disturbance for the first week of differentiation. The following two weeks of differentiation 50% of the starting volume of fresh factory media was added once per week. From the third week of differentiation half-media changes were done, until the production and release of (CD14+) monocytic progenitors in the supernatant can be detected. From this point on, complete media change with fresh factory media was performed twice a week.

Harvesting of Monocytes

Monocytes were collected from the supernatant by centrifugation (4 minutes, 300 g), cells were re-suspended, counted and quality control (CD68, Ki67, CD11b and CD14) of marker expressions by flow cytometry was performed weekly. Monocytic progenitors were transferred to differentiation media and differentiated to macrophages or in co-culture with neurons to microglia.

Differentiation of Macrophages

According to application requirements macrophages were either directly differentiated in the required plate format or pre-differentiated on Upcell™ plates for 6 days and then re-plated, following the manufacturer protocol, to the final plate format one day prior to assay start. For differentiation cells were either cultured in XVIVO 15 (supplemented with 2 mM Glutamax, 1% Penstrep and 10-200 ng/ml M-CSF) or RPMI1640 (supplemented with 1% Penstrep and 10-200 ng/ml M-CSF or 1-10% fetal bovine serum). Media was changed 3 days after plating; cells were differentiated for 7 days.

Polarization of Macrophages

For polarization of macrophages to pro-inflammatory (M1) or regulatory phenotype (M2), cells were cultured in XIVIVO15 media supplemented with 2 mM Glutamax, 1% Penstrep, 5-100 ng/ml GM-CSF and 1-100 ng/ml INFy (M1) or supplemented with 2 mM Glutamax, 1% Penstrep, 5-100 ng/ml M-CSF and 1-100 ng/ml IL-4 (M2), respectively for the desired polarization period.

Generation of Microglia Like Cells in Neuronal Co-Culture

For differentiation of monocytes to microglia like cells, monocytes were plated on pre-differentiated neurons and co-cultured for two weeks prior to analysis.

Neuron Generation

Neurons were differentiated as described in WO2017081250 and large stocks were frozen at day 21. Two weeks prior to initiation of the co-cultures, neurons were thawed and seeded at a density of 50-200000 cells per cm² in N2/B27 media containing BDNF, GDNF, cAMP, ascorbic acid and 10 μM ROCK inhibitor (Y27632, Callbiochem) on cell culture vessels pre-coated with 5 ug/ml recombinant humanLaminin-521 (BNioLamina). Media was changed every 3 days (without ROCK inhibitor for the further course of neuronal maturation).

### Co-Culture

Freshly harvested monocytic progenitors were plated on top of mature neurons in N2 media (consisting of: Advanced DMEM F-12, N2 supplement, Glutamax, 50 μM Mercaptoehtanol, 1% P/S and 1-100 ng/ml GM-CSF and 1-500 ng IL-34). Microglia cells were matured in co-culture for 14 days with media change twice a week.

Monocyte Collection and Intermediate Storage in Suspension Cultures

Freshly harvested monocytic progenitors were collected and cultured over several weeks in suspension cultures named “Spinner” in XVIVO15 media (Lonza) supplemented with 2 mM Glutamax, 1% Penicillin/Streptomycin, 50 ug/ml Mercaptoethanol, M-CSF (20-200 ng/ml) and IL3 (1-50 ng/ml). Cell number was adjusted to 0.5-2 mio/ml, media exchange was performed twice a week, cells were re-suspended, counted and quality controled (CD68, Ki67).

### Example 1

### Modified Stem Cell Maintenance Facilitates Blood Factory Differentiation and Increases Yields of Monocytes

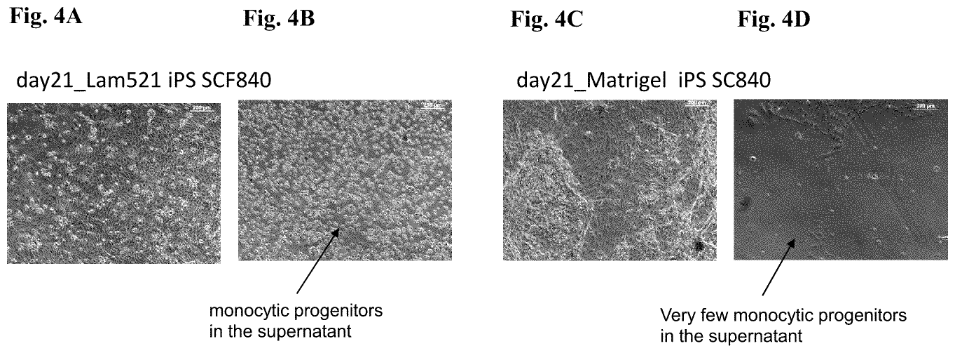

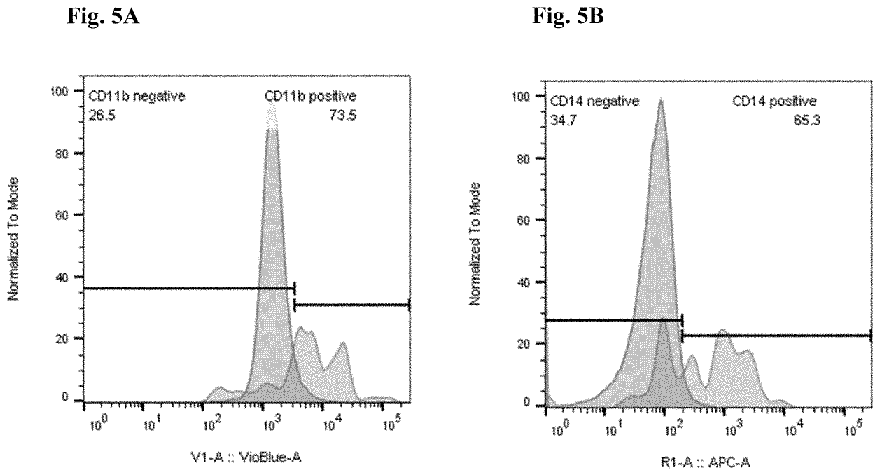

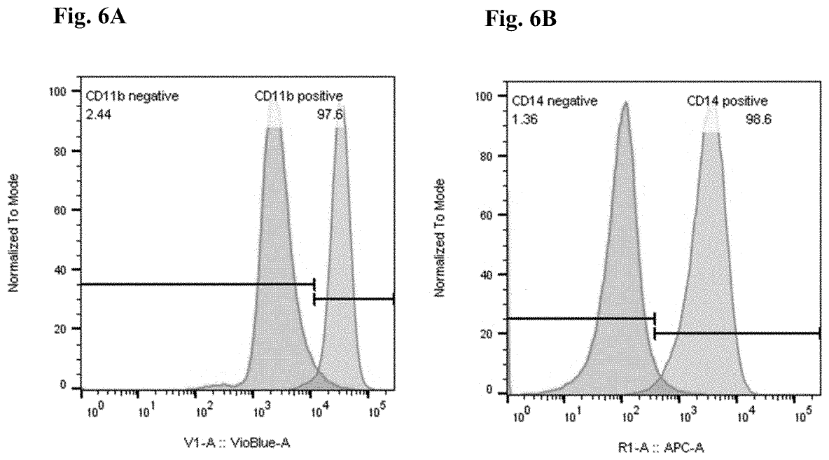

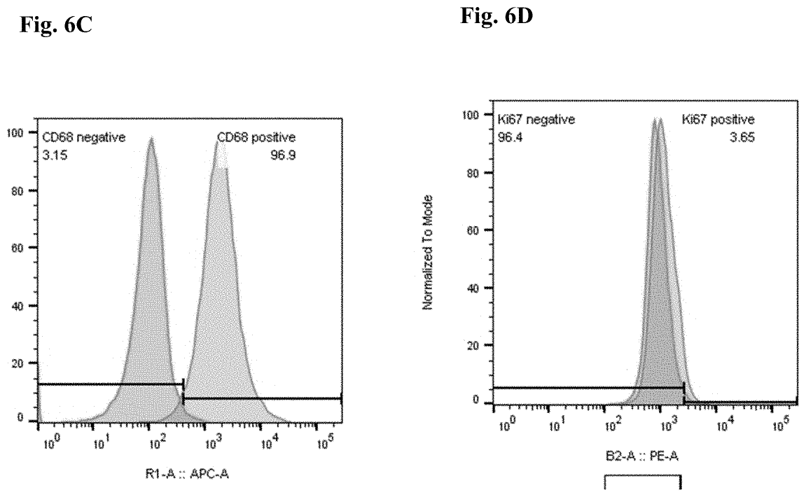

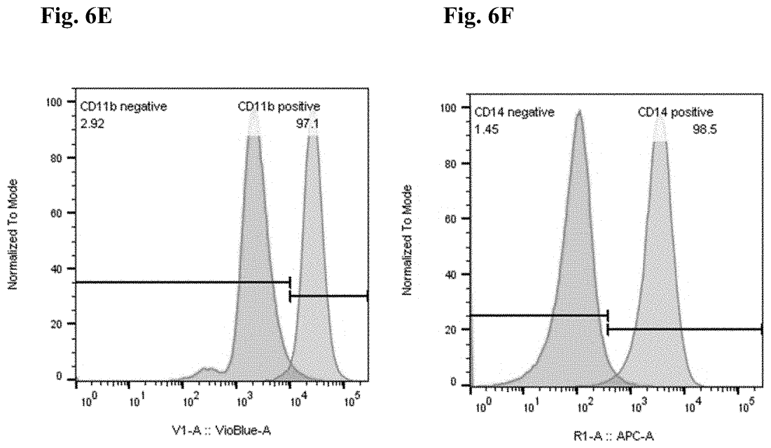

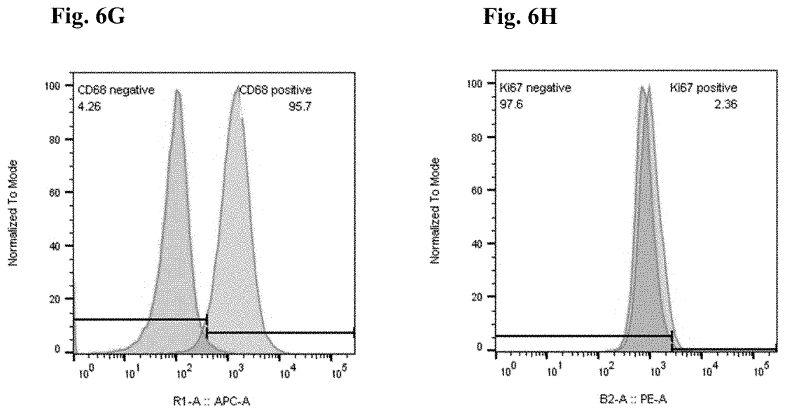

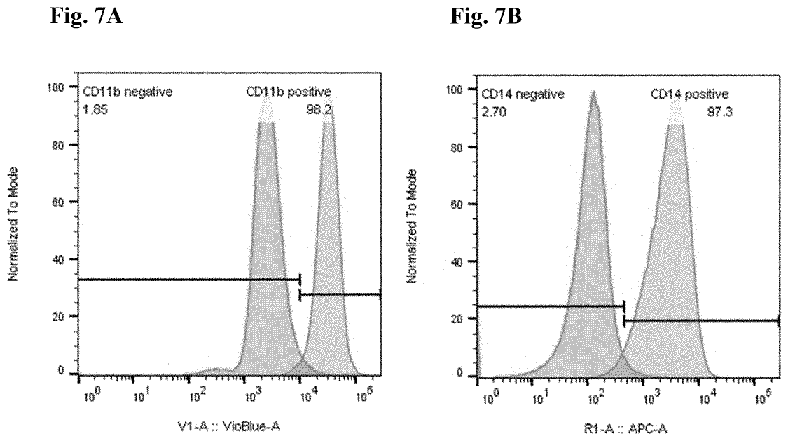

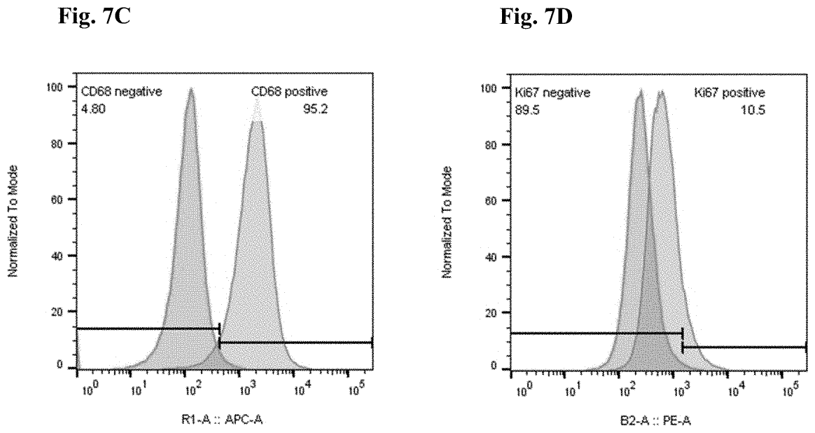

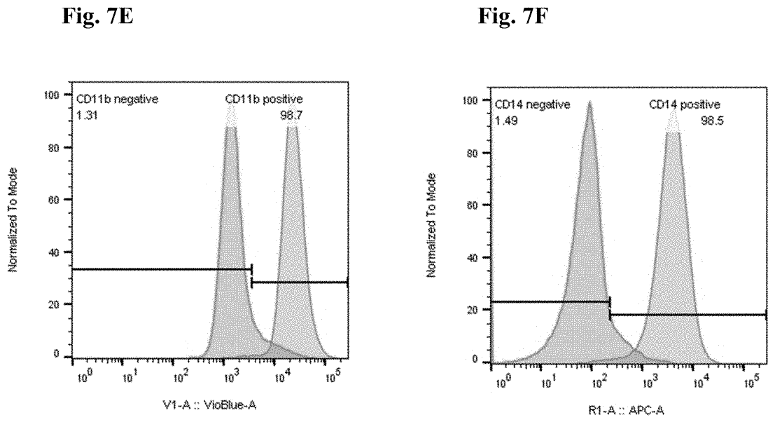

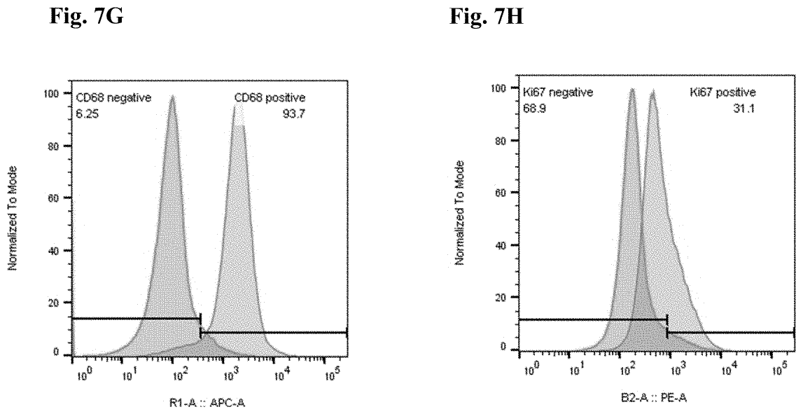

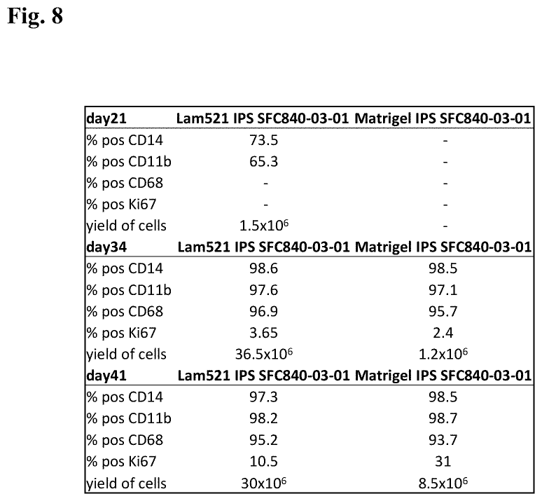

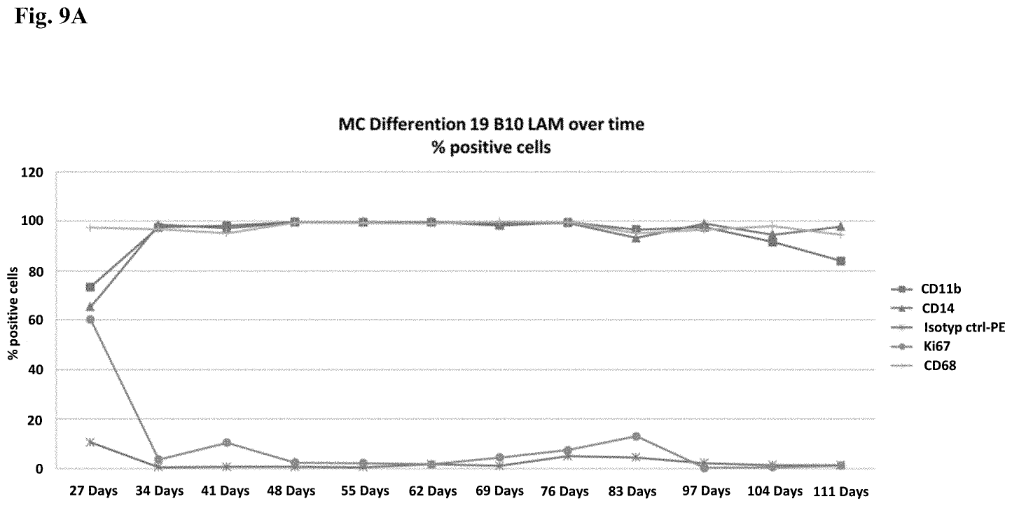

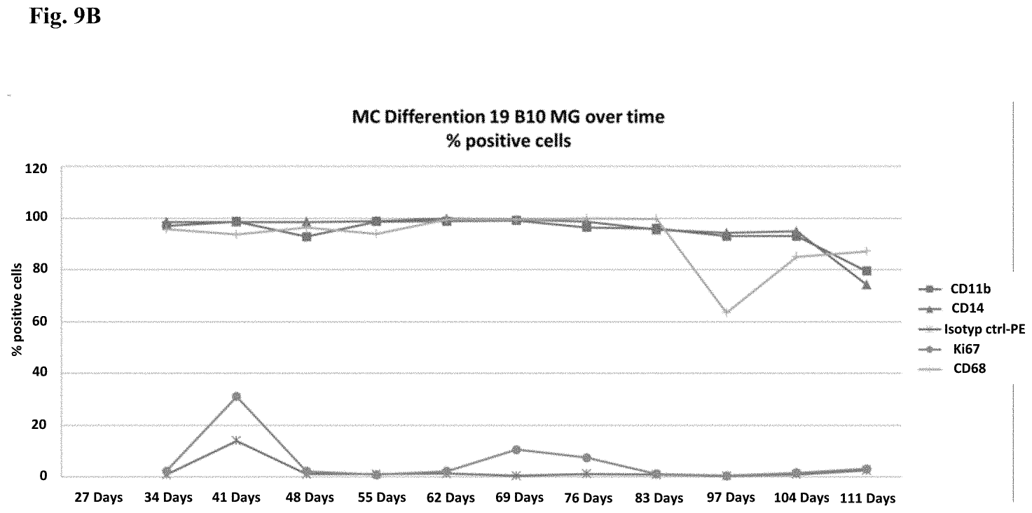

Induced pluripotent stem cells were cultured in feeder free conditions and differentiated to blood factories as described above. The substitution of matrigel by Laminin-521 coating substrate in the stem cell maintenance culture decreased the differentiation time of blood factories. Blood factories derived from iPSC cultured on Laminin-521 started to produce monocytic progenitors at day 21 of differentiation, while there was no monocytic progenitors released in the supernatant by blood factories derived from iPSC cultured on matrigel until day 34 of differentiation (FIG. 4). In line with the earlier release of macrophages also marker gene expression of monocytes increased earlier during the differentiation process and the weekly harvest yields were significantly higher in the blood factories derived from iPSC cultured on Laminin-521 (FIG. 5-9). This observation points to the high relevance of iPSC maintenance conditions for an efficient differentiation process.

### Example 2

### iPSC-Derived Monocytic Progenitors Differentiate into Macrophages with Comparable Marker Pattern to Primary Human Macrophages Cultured

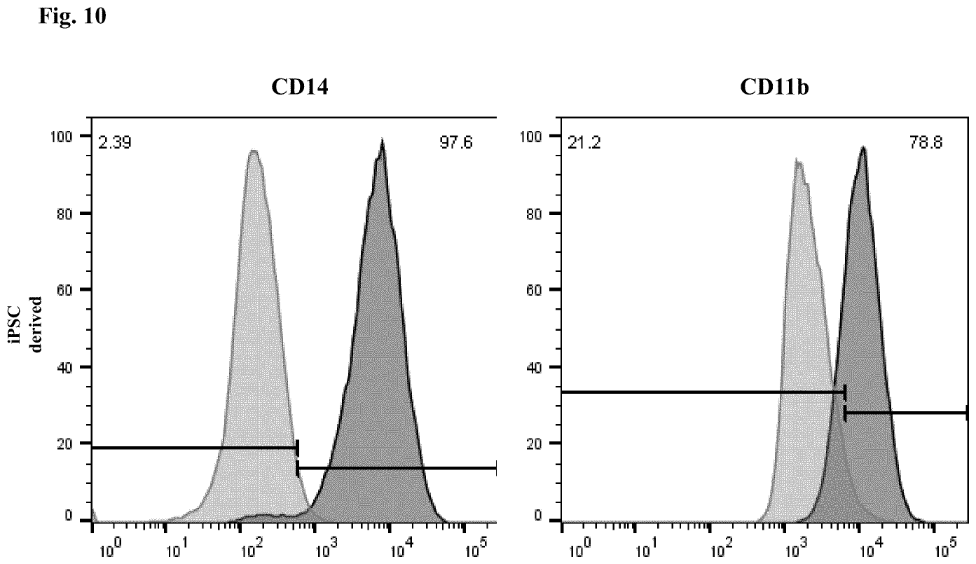

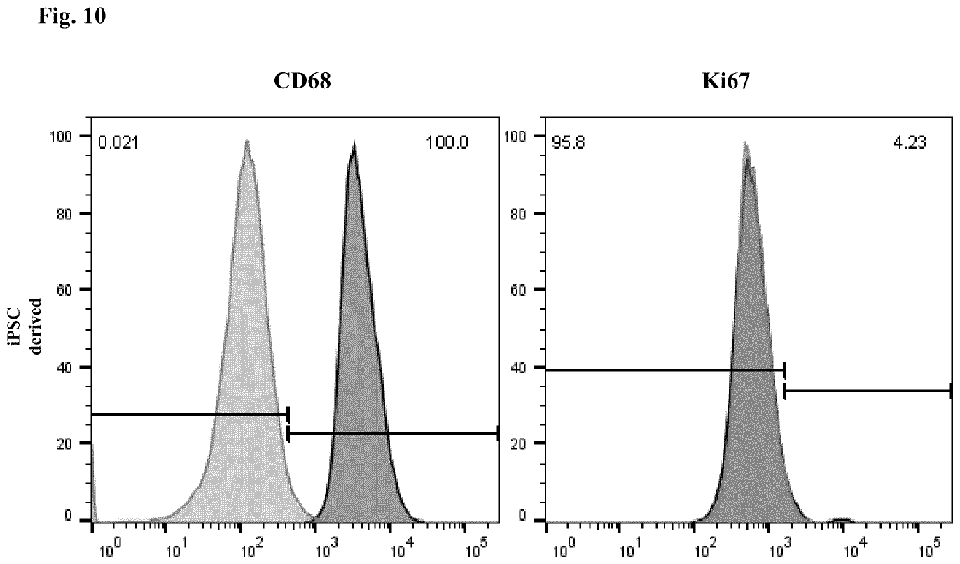

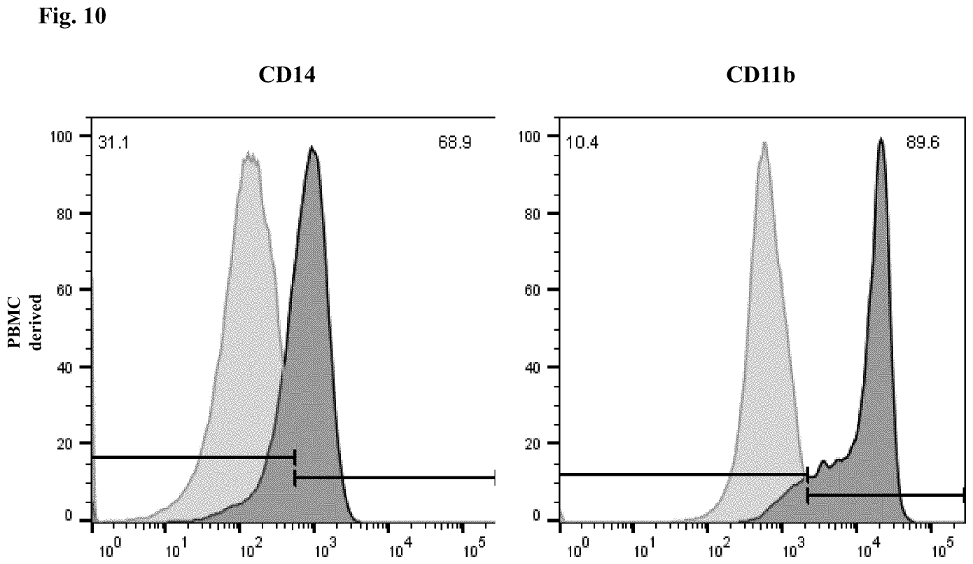

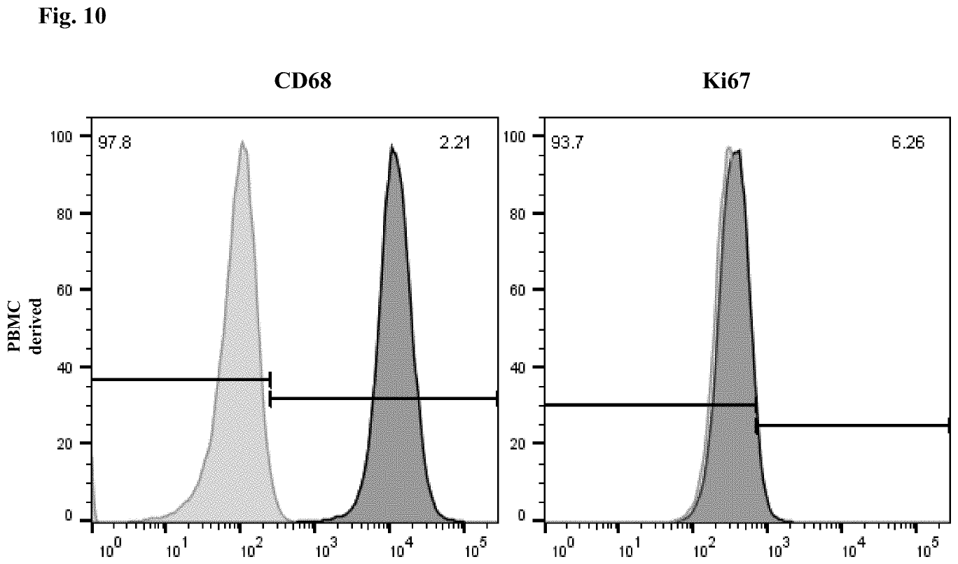

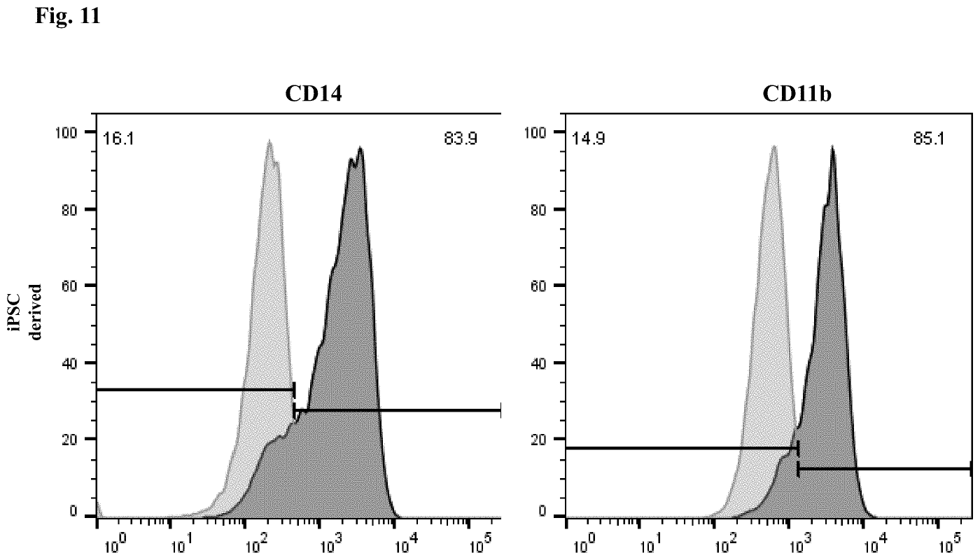

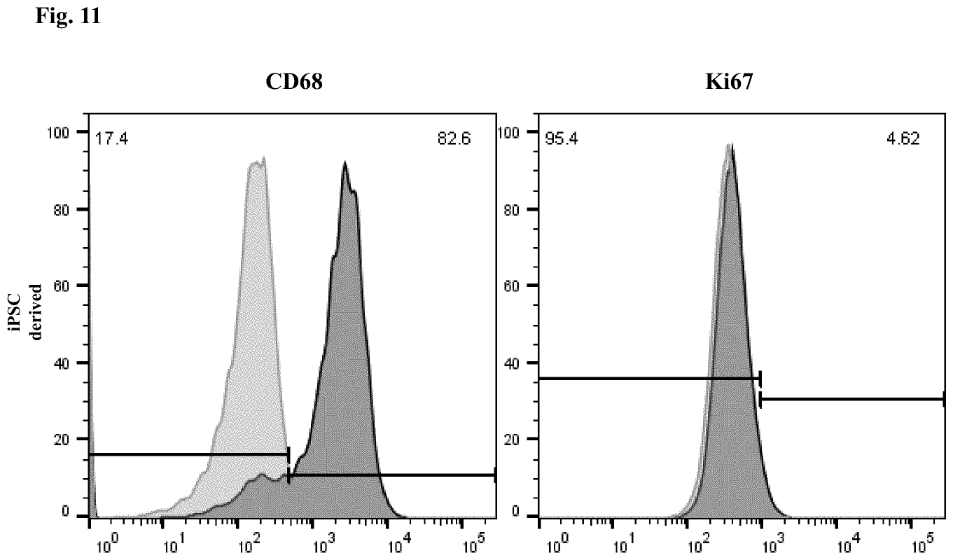

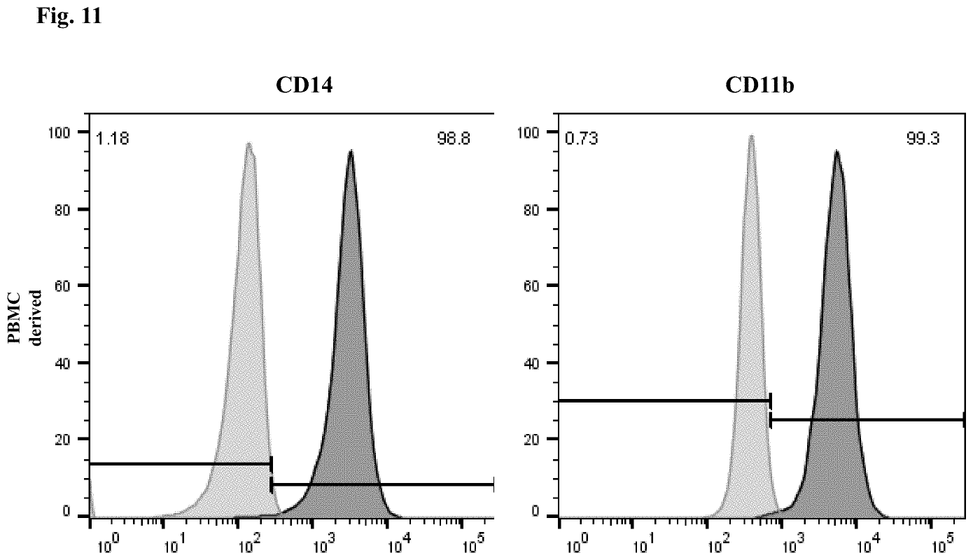

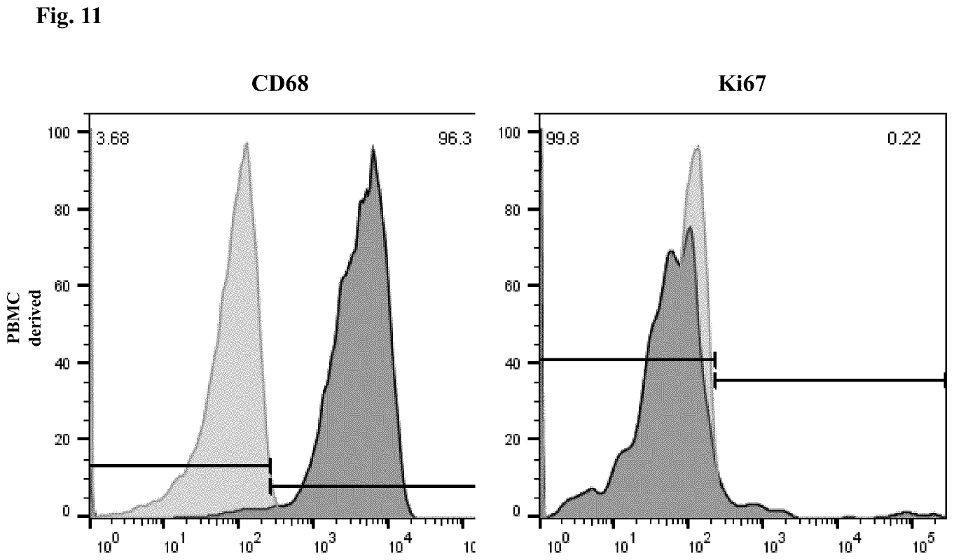

To compare iPSC-derived macrophages with primary macrophages, monocytic progenitors derived from iPS cells and CD14 positive blood monocytes obtained from LONZA were differentiated into macrophages as described above. Marker gene expression in starting population (monocytes/FIG. 10) and in macrophages (FIG. 11) was assessed by flow cytometer for CD14, CD11b, CD68 and Ki67. Monocytes derived from iPS cells had higher levels of CD14 and weaker expression of CD11b, but an overall comparable marker expression pattern (FIG. 10). Macrophages differentiated from both sources displayed similar marker patterns as well (FIG. 11), indicating that iPSC-derived monocytic progenitors and macrophages are a valid alternative source for in vitro models of myeloid biology.

### Example 3

### Enhanced Culture Conditions of Blood Factories Improve Adherence of EBs and Blood Factory Stability

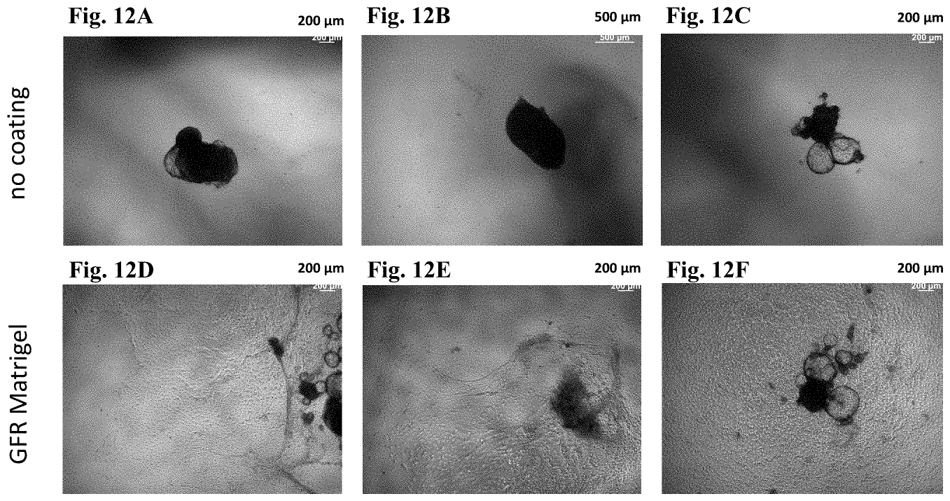

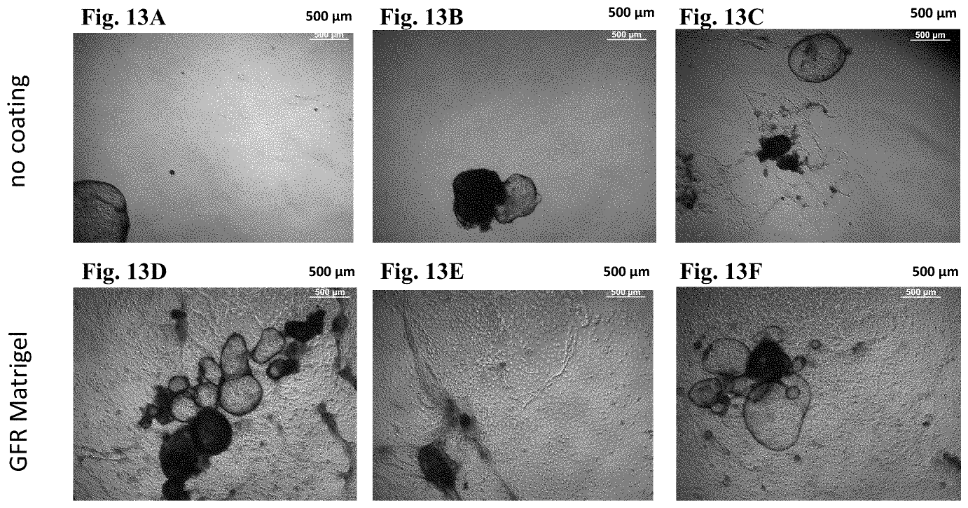

EBs from three iPSC lines derived from different donors were generated as described above and plated either on culture vessels pre-coated with growth factor reduced matrigel or on untreated culture vessels and adherence and culture stability was monitored visually over the differentiation period (FIGS. 12 and 13). EBs from all donors adhered better to growth factor reduced matrigel coated plates and more cell outgrowth from the EBs was observed on coated plates. This protocol change increases the culture stability and thereby increases the long term culture success rate.

### Example 4

### The New Culture Protocol Allows Generation of Functional Macrophages from Different iPS Cell Lines

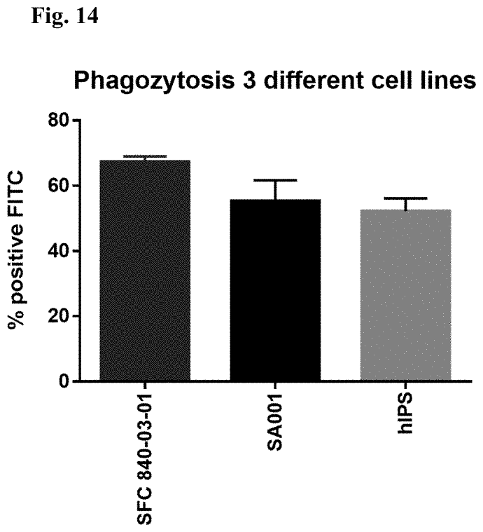

Monocytic progenitors and Macrophages were derived from three different iPS cell lines as described above. To assess macrophage functionality, the phagocytotic capacity of these macrophages was tested by incubating them for 120 minutes with pHrodo green labeled zymosan, and a subsequent flow cytometric analysis to detect green cells (FIG. 14). After 120 min incubation about 60% of the cells had taken up zymosan particles as measured by green fluorescence. Only minor differences between the three different iPSC donors were observed (FIG. 14), which underlines the robustness of the differentiation protocol.

### Example 5

### Microglia Differentiation in Co-Culture with Neurons

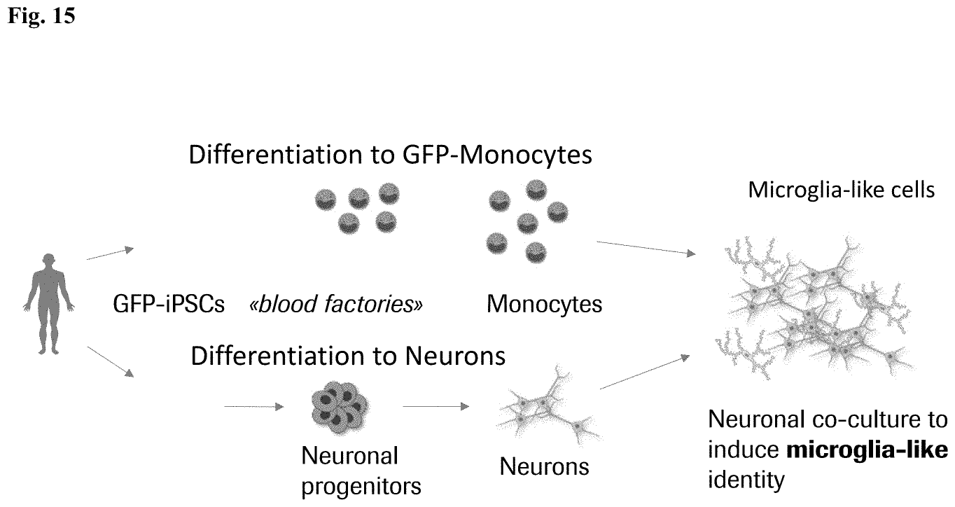

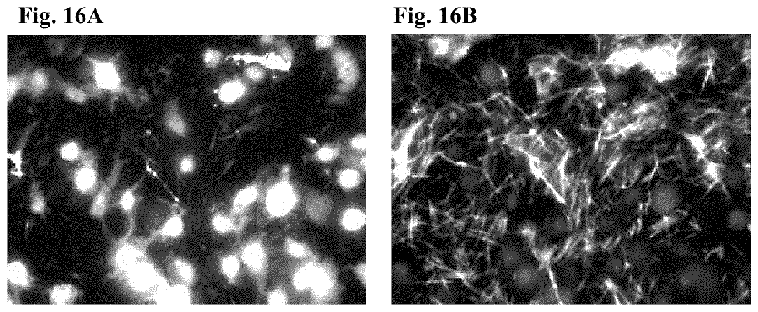

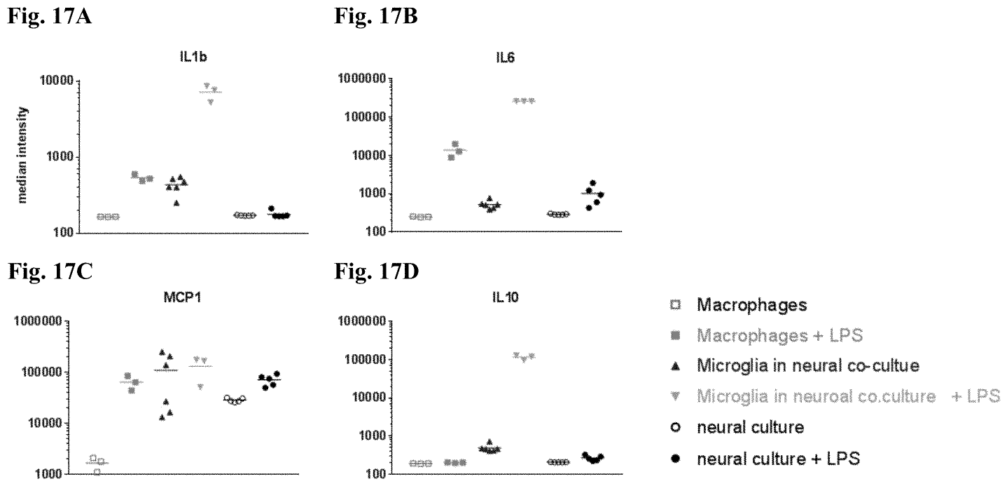

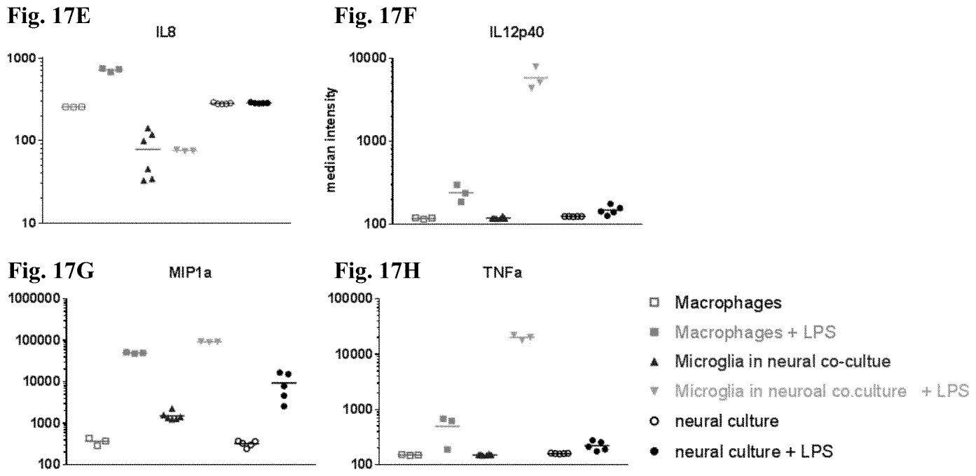

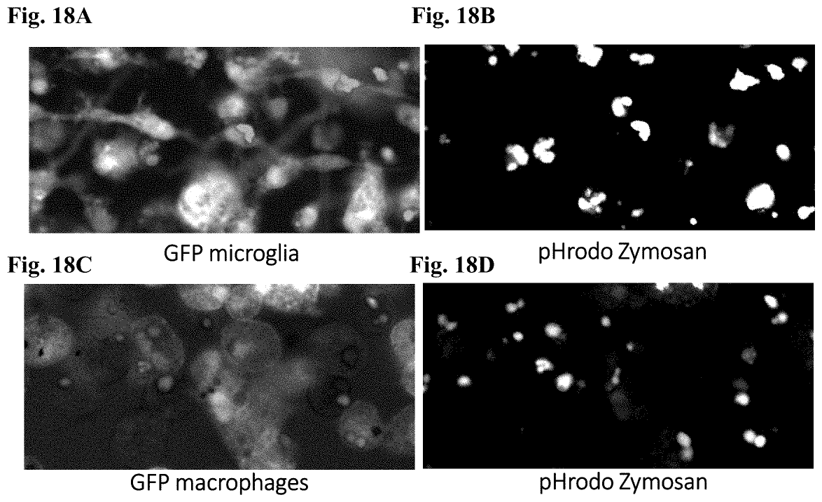

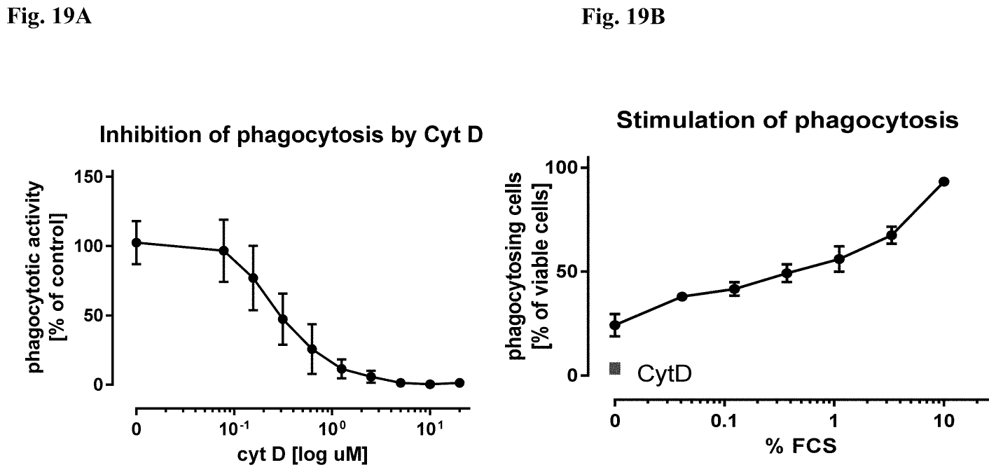

Monocytic progenitors derived from iPS cells as described above can be co-cultured with human iPSC-derived neurons in order to differentiate them into microglia-like cells (Haenseler et al. 2017a) (overview FIG. 15). Here we shortened the published protocol, when seeding monocytic progenitors on neurons re-thawed at week 3 of differentiation (WO2017081250). The use of this frozen neuronal stocks allows a higher flexibility and throughput in the experimental co-culture design. By using iPS cells that have a stable expression of GFP, GFP positive monocytic progenitors and microglia-like cells can be generated, which facilitates the live cell imaging and microglia detection in those co-cultures (FIGS. 15 and 16). Interestingly, upon LPS stimulation microglia in co-culture showed differences in the cytokine release pattern, when compared to mono-culture macrophages and neuronal-monocultures (FIG. 17), indicating the potential use as neuro-inflammation model. In contrary to the alterations in cytokine release, phagocytosis measurement by using pHrodo red zymosan in combination with GFP positive macrophages and microglia in a high content imaging setup reveals similar uptake of zymosan particles by macrophages and microglia (FIG. 18). Employing high content imaging and miniaturization of the assay to 384 wells, this setup can be used for screening of modulators of phagocytosis in macrophages and microglia (FIG. 19), here shown by dose dependent inhibition of phagocytosis with cytochalasin D and dose dependent stimulation with serum incubation.

### Example 6

### Collection of Monocytic Progenitors in Suspension Culture for Large Scale Screening Campaigns

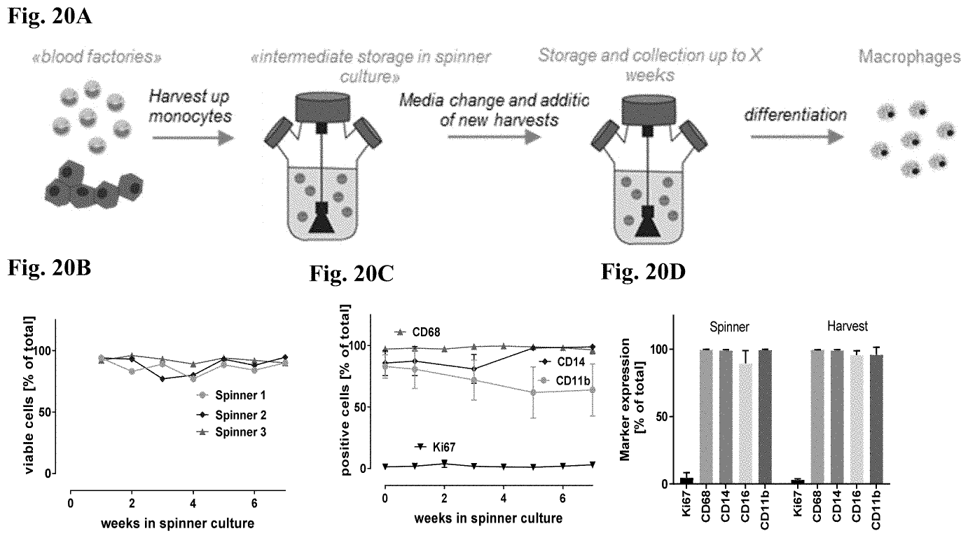

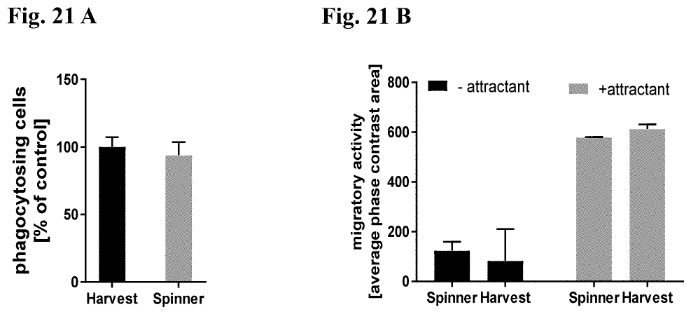

Monocytic progenitors were harvested from blood factories and collected for several weeks in suspension cultures (FIG. 20A). Viability of monocytic progenitors as well as marker expression stayed constant for at least 6 weeks in suspension cultures (FIGS. 20B and C). When monocytes were taken at different time points from suspension cultures and differentiated to macrophages the maker expression of the resulting macrophages showed no difference between the cells derives from suspension culture compared to the cells derived directly after harvesting (FIG. 20D). The possibility of generating large homogenous populations of monocytic progenitors is well suited for screening applications; therefore cells stored in such suspension cultures should give rise to macrophages that have comparable functional characteristics to directly differentiated macrophages. To assess macrophage functionality, the phagocytotic capacity of macrophages derived from suspension cultures (“Spinner”) and fresh harvests (“Harvest”) was tested by incubating them for 120 minutes with pHrodo red labeled zymosan, and a subsequent high content based analysis to detect phagocytosing cells (FIG. 21A). No differences between the two conditions were observed (FIG. 21 A). A second functional property of macrophages is the ability to migrate towards a chemoattractant; we assessed this for the two populations of macrophages by using the IncuCyte transwell assay (Essen Bioscience) and the chemoattractant C5a. Also in this setup cells derived from suspension culture (“Spinner”) showed no significant difference compared to cells, which were differentiated from freshly harvested monocytic progenitors (“Harvest”), in their migratory behavior (FIG. 21B). The comparable functional properties and marker expression confirms the phenotype and usability of the cells derived from the suspension cultures for large scale functional and phenotypic assays.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for producing monocytic progenitor cells, the method comprising the step of:
a) plating pluripotent stem cells in a pluripotency medium on a cell culture support coated with laminin;
b) harvesting the pluripotent stem cells and contacting the pluripotent stem cells with a mesoderm induction medium in suspension culture;
c) plating the cells on a cell culture support suitable for attachment of the cells; and
d) harvesting monocytic progenitor cells from the cell culture supernatant.

2. The method of claim 1, wherein the laminin in step a) comprises the laminin subunit alpha-5, in particular wherein the laminin in step a) comprises the laminin subunits alpha-5, beta-2 and gamma-1.

3. The method of claim 1 or 2, wherein the cells are contacted in step b) with a defined medium comprising BMP4.

4. The method of any one of claims 1 to 3, wherein the cells are contacted in step b) with a defined medium comprising VEGF.

5. The method of any one of claims 1 to 4, wherein the cells are contacted in step b) with a defined medium comprising SCF.

6. The method of any one of claims 1 to 5, wherein the cells in step b) form embryoid bodies (EBs).

7. The method of any one of claims 1 to 6, wherein the cell culture support in step c) is coated with a basement membrane biomaterial.

8. The method of any one of claims 1 to 7, wherein the cells in step c) are contacted with a myeloid maturation medium.

9. The method of any one of claims 1 to 8, wherein the myeloid maturation medium comprises M-CSF.

10. The method of any one of claims 1 to 9, wherein the myeloid maturation medium comprises IL-3.

11. The method of any one of claims 1 to 10, further comprising step e) differentiating the harvested monocytic progenitor cells into macrophages.

12. The method of claim 11, wherein the cells in step e) are plated onto a non-coated tissue culture support.

13. The method of claims 1 to 10, further comprising step e) differentiating the harvested monocytic progenitor cells into microglia.

14. An adherent large scale cell culture for producing monocytic progenitor cells, wherein the adherent cell culture is capable of producing at least about 100'000 monocytic progenitor cells per cm² of cell culture area per week.

15. The adherent large scale cell culture of claim 14 produced by the method of any one of claims 1 to 13 steps a) to c).
